(19) United States
(12) Patent Application Publication
(10) Pub. No.: US 2012/0253314 A1
(43) Pub. Date: Oct. 4, 2012

(54) PALM-CONTROLLED INJECTORS

(76) Inventors: Ziv Harish, Tenafly, NJ (US); Saac Rubinstein, Haworth, NJ (US); Russ Weinzimmer, Milford, NH (US); Ehud Arbit, Englewood, NJ (US)

(21) Appl. No.: 13/076,405

(22) Filed: Mar. 30, 2011

(51) Int. Cl. A61M 5/42; G09B 23/28; A61M 5/31

(52) U.S. Cl. 604/506; 604/182; 604/112; 434/262

(57) ABSTRACT

A palm-controlled device is disclosed for injection of a substance into an organism. The device includes a palm-receiving surface for receiving a palm of a hand, the palm receiving surface being shaped so that the palm is substantially parallel to a surface of an injection site of the organism while operating the device. The palm receiving surface can also be shaped so that the palm is substantially perpendicular to a surface of the injection site. The device also includes an injector having at least one pre-filled syringe, the palm-receiving surface being cooperative with the injector such that when pressure is applied to the palm-receiving surface, the injector is actuated so as to inject contents of the at least one pre-filled syringe. Actuation of the injector is by gradual pressure; not sudden motion. Single or multiple doses of same or different medications can be included in a single injector.

### FIELD OF THE INVENTION

This Invention generally relates to administration of substances, and particularly to devices for injection of substances into an organism.

### BACKGROUND OF THE INVENTION

Current auto-injectors are pen-shaped pre-loaded impact-activated syringes. These auto-injectors have concealed needles to help overcome the common fear of needles that may inhibit their use in an emergency situation. The most commonly used auto-injector is EpiPen®, which is an auto-injector that is pre-loaded with a standard dose of epinephrine, which reverses anaphylactic reactions commonly induced by food, drug, or insect venom. Further, auto-injectors can be pre-loaded with other medications. For example, some countries have stockpiles of such auto-injectors for their military and their citizens in preparation for chemical warfare, especially to protect against nerve gas. Prior to use of the auto-injector, a safety cap must be removed to allow the needle to penetrate into the thigh. To distinguish between the needle-end and the safety-cap-end of the auto-injector, each end has a different color and shape.

### Pitfalls of Current Auto-Injectors

### 1. Counter-Intuitive Shape

Even though patients and caregivers are given detailed in-person explanations and demonstrations of the proper use of an EpiPen®, as well as being shown a training DVD, and are provided with an EpiPen® Trainer for practice, it has been observed that about 50% of patients and caregivers fail to demonstrate correct use of the auto-injector during follow-up visits. In an emergency situation, those mistakes would render the EpiPen® ineffective. Further, it has also been observed that physicians and nurses are among the caregivers that fail to correctly use the EpiPen®, even in a calm office environment. For example, one common mistake that is observed is holding the auto-injector upside-down, resulting in accidentally injecting the loaded dose of medication into the thumb of the caregiver or the patient. This common mistake is an unintended result of the design of the EpiPen®. Since the EpiPen® resembles a pen or marker, the user anticipates that the end with the safety cap is the needle end of the auto-injector. However, the end of the EpiPen® with the safety cap is the end that is opposite to the end with the needle. Consequently, many EpiPen® users fail to perform the required flip of the device, and consequently press on the needle end, thereby injecting the epinephrine into their own thumb. Even if this is the patient's own thumb, there is no absorption of epinephrine from the thumb, consequently depriving the patient of the benefit of the epinephrine. Moreover, the Epinephrine severely constricts the arterial supply to the thumb, and may result in local damage. Reportedly, a child's life was recently lost due to an anaphylactic shock when a parent wasted the only available EpiPen® dose by injecting it into his/her own thumb.

### 2. Intimidating or Frightening Recommended Mode of Administration

The recommended motion for administering is shown on the label of the EpiPen® as a stabbing motion into the leg, and is described on the EpiPen® device as: “Jab black tip firmly into outer thigh so it ‘clicks’ AND HOLD on thigh approx. 10 secs.”. And on the new version of the EpiPen®, the label shows a stabbing motion into the leg, and instructs the user to: “Swing and firmly push”. Consequently, many children and adults show and express reluctance to use the EpiPen® because of the frightening stabbing nature of the recommended mode of administration.

### 3. Incomplete Administration

There is no indication when the EpiPen® has completed the injection. Many patients and caregivers apply the EpiPen® in a fast downward swinging motion, and then incorrectly lift it off the thigh too soon, thereby disengaging the needle of the EpiPen® possibly not allowing enough time for a complete dose of epinephrine to be delivered.

### 4. Bulkiness

A single EpiPen® measures 6.5″ in length, and 1.25″ in diameter. Routinely, patients who need epinephrine to be always available to save their lives when the need arises, are often instructed by a physician to carry two EpiPens® at all times, because occasionally, a patient may need more than one EpiPen® to reverse a severe allergic reaction. However, only 20-30 percent of patients will actually carry two EpiPens®. Further, the bulkiness of the EpiPen® often deters individuals from carrying even a single EpiPen®.

### SUMMARY OF THE INVENTION

One general aspect of the invention is a palm-controlled device for injection of a substance into an organism. The device includes a palm-receiving surface for receiving a palm of a hand, the palm receiving surface being shaped so that the palm is substantially parallel to a surface of an injection site of the organism while operating the device; and an injector having at least one pre-filled syringe, the palm-receiving surface being cooperative with the injector such that when pressure is applied to the palm-receiving surface, the injector is actuated so as to inject contents of the at least one pre-filled syringe into the injection site of the organism.

In some embodiments, the palm-receiving surface is ergonomically shaped so as to receive at least a palm.

In some embodiments, the injector has an injector base through which the needle is moved when performing an injection. In further embodiments, the injector base includes a concave surface which substantially conforms to a convex surface including the injection site. In other further embodiments, the injector base include features for activating a “pain gate” prior to penetration of the injection needle into the injection site. In still further embodiments, the features for activating a “pain gate” include at least one of: a plurality of protrusions extending out from the surface of the injector base so as to provide a plurality of localized pressure areas to a region surrounding an injection site before and during penetration of the needle into the injection site; at least one annular ridge of the surface of the injector base so as to provide at least one localized pressure area to a region surrounding at least one injection site before and during injection of at least one needle into at least one respective injection site; and a substantially flat surface of the injector base so as to provide a continuous area for applying pressure to a region surrounding an injection site before and during injection into the injection site. In further embodiments, the plurality of protrusions include at least one of: balls, cubes, prisms, pyramids, bumps, cylinders, blunt probes. In other further embodiments, the plurality of protrusions are arranged in at least one of: a regular array; a random spatial distribution; a concentric arrangement.

In some embodiments, the injector is capable of injecting at least one pre-loaded substance.

In some embodiments, the injector is capable of injecting a plurality of pre-loaded substances, the injecting occurring sequentially or simultaneously.

In some embodiments, the injector can automatically retrieve the needle after injection of the substance.

In some embodiments, the injector can include at least one safety mechanism for ensuring safe operation of the device.

In some embodiments, the injector includes a plurality of safety mechanisms, operation of each safety mechanism allowing injection of a single substance corresponding to a respective safety mechanism.

In some embodiments, the injector includes an element for transmitting manual pressure applied by at least the palm of the hand to a syringe including the injection needle so as to cause the injection needle to penetrate into the injection site.

In some embodiments, the injector includes an element for transmitting manual pressure to a trigger that allows automatic force to be applied to a syringe including the injection needle so as to cause the injection needle to penetrate into the injection site.

In some embodiments, the injector includes an element for transmitting manual pressure applied by at least the palm of the hand to a syringe including a plunger so as to cause the plunger to inject the substance into the injection site.

In some embodiments, the injector includes an element for transmitting manual pressure to a trigger that allows automatic force to be applied to a plunger so as to cause the plunger to inject the substance into the injection site.

In some embodiments, injection of a substance follows after penetration of the injection needle. In some embodiments, injection of a substance proceeds while penetration of the injection needle proceeds.

In some embodiments, the injector includes: a signaling element capable of indicating when a complete dose of a substance has been injected, by at least one of: visual signaling, audible signaling, tactile signaling.

In some embodiments, the injector includes: apparatus capable of enabling both installing and removing a pre-filled syringe including the injection needle.

In some embodiments, the injector includes: apparatus capable of automatically withdrawing the injection needle back into the injector after injection of the substance. In further embodiments, the injector includes a syringe having the injection needle, a cylinder, and a plunger, and the apparatus includes an element that interlocks the plunger with the cylinder so as to enable withdrawal of the injection needle by simultaneous withdrawal of the plunger and the cylinder.

In some embodiments, the injector includes a syringe having a retractable injection needle that is automatically retracted into the syringe after injection of the substance.

Another general aspect of the invention is a palm-controlled device for injection of a substance into an organism. The device includes: a palm-receiving surface for receiving a palm of a hand, the palm receiving surface being shaped so that the palm is substantially parallel to a surface of an injection site of the organism while operating the device; and an injector having apparatus capable of accepting and releasing at least one pre-filled syringe, the palm-receiving surface being cooperative with the injector such that when pressure is applied to the palm-receiving surface, the injector is actuated so as to inject contents of at least one accepted pre-filled syringe into the injection site of the organism.

In some embodiments, the palm-receiving surface is ergonomically shaped so as to receive at least a palm.

In some embodiments, the injector has an injector base through which the needle is moved when performing an injection. In further embodiments, the injector base includes a concave surface which substantially conforms to a convex surface into which the injection needle is to be injected. In other further embodiments, the injector base include features for activating a “pain gate” prior to injection of the injection needle.

In some embodiments, the injector is capable of accepting at least one pre-loaded syringe.

In some embodiments, the injector can automatically withdraw the needle after injection of the substance.

In some embodiments, the injector includes an element for transmitting manual pressure applied by at least the palm of the hand to the pre-filed syringe including the injection needle so as to cause the injection needle to penetrate into the injection site.

In some embodiments, the injector includes an element for transmitting manual pressure applied to a trigger that allows automatic force to be applied to the pre-filled syringe including the injection needle so as to cause the injection needle to penetrate into the injection site.

In some embodiments, the injector includes an element for transmitting manual pressure applied by at least the palm of the hand to a syringe including a plunger so as to cause the plunger to inject the substance into the injection site.

In some embodiments, the injector includes an element for transmitting manual pressure applied to a trigger that allows automatic force to be applied to a plunger so as to cause the plunger to inject the substance into the injection site.

In some embodiments, injection of a substance follows penetration of the injection needle. In some embodiments, injection of a substance proceeds while penetration of the injection needle proceeds.

In some embodiments, the injector includes: a signaling element capable of indicating when a complete dose of a substance has been injected, by at least one of: visual signaling, audible signaling, tactile signaling.

Another general aspect of the invention is a palm-controlled device for injection of a substance into an organism. The device includes: a palm-receiving surface for receiving pressure directly applied by at least the palm of a hand, the palm of the hand having a surface normal vector, the pressure being characterized by a pressure vector, the surface normal vector being substantially parallel to the pressure vector; and an injector having an injection needle, the injector being characterized by an injection vector that is co-linear with the injection needle, the injection vector being substantially parallel to the surface normal vector.

Another general aspect of the invention is a palm-controlled device for injection of a substance into an organism. The device includes: a palm-receiving surface for receiving pressure directly applied by at least the palm of a hand, the palm of the hand having a surface normal vector, the pressure being characterized by a pressure vector, the surface normal vector being substantially parallel to the pressure vector; and an injector having apparatus capable of accepting and releasing at least one pre-filled syringe, the injector being characterized by an injection vector that is co-linear with an injection needle of the pre-filled syringe when accepted by the apparatus, the injection vector being substantially parallel to the surface normal vector.

Another general aspect of the invention is a palm-controlled training device for teaching injection of a substance into an organism. The device includes: a palm-receiving surface for receiving a palm of a hand, the palm receiving surface being shaped so that the palm is substantially parallel to a surface of a potential injection site of the organism while operating the device; and an injector having at least one dummy syringe, the palm-receiving surface being cooperative with the injector such that when pressure is applied to the palm-receiving surface, the injector is actuated so as to simulate injection of contents of the at least one dummy syringe into the potential injection site of the organism.

Another general aspect of the invention is a method for injection of a substance into an organism. The method includes: using an open palm of a hand to activate an injector to inject the substance into an injection site of the organism.

In some embodiments, the palm is substantially parallel to a surface of an injection site of the organism.

In some embodiments, when the injector is activated, contents of at least one pre-filled syringe is injected into an injection site of the organism.

Another general aspect of the invention is a palm-controlled device for injection of a substance into an organism. The device includes: a palm-receiving surface for receiving a palm of a hand, the palm receiving surface being shaped so that the palm is substantially perpendicular to a surface of an injection site of the organism while operating the device; and an injector having apparatus capable of accepting and releasing at least one pre-filled syringe, the palm-receiving surface being cooperative with the injector such that when pressure is applied to the palm-receiving surface, the injector is actuated so as to inject contents of at least one accepted pre-filled syringe into the injection site of the organism.

The palm-activated injectors of the invention are shaped so as to appear friendly and non-threatening, and are adapted so as to encourage a gentle pressing action for triggering the injection mechanism of the palm-activated injector. Moreover, the shape is conducive to proper application, i.e., proper application means placement of the palm-activated injector prior to actuation of the palm-activated injector, with the needle away from the operating hand, so as to ensure injection into the intended injection site, and NOT inadvertent injection into one's thumb or hand.

Further, the shape of the palm-activated injector suggests application in the correct orientation, and consequently, the possibility of shape-induced confusion regarding application orientation is significantly reduced. In addition, the orientation does not need to change at any time during operation of the device.

Some embodiments of the palm-activated injector are convenient to carry, including carrying in a small pocket, even when containing multiple doses of medication(s).

In some embodiments, the concealed needles of the palm-activated injector of the invention are automatically injected only after intentionally deactivating a safety mechanism, such as removing a safety pin, which allows activation of a trigger mechanism, thereby initiating injection of a medication.

In preferred embodiments of the invention, needle(s) extend out of the injector only during active administration of the medication, and consequently, needle injuries are unlikely to occur. For example, some embodiments of the palm-activated injector of the invention include a self-withdrawing needle that protects the user from accidental needle-stick after injection. Other embodiments include a self-withdrawing syringe, which concomitantly withdraws the needle upon completion of the injection.

The injectors of the invention have a non-threatening shape that is not reminiscent of known syringes. Further, the shape of the injectors encourages a more gentle approach of the injecting device to the recipient of the injection. By contrast, since many non-health professionals need to inject themselves and/or their dependents, they are often reluctant to perform the injection using known injectors and known syringes, because the stabbing motion of the injection is commonly perceived to be aggressive and/or threatening by both the caregiver and by the recipient.

Some embodiments of the injector of the invention incorporate “pain gate” features that reduce perceived discomfort of the injection performed by the injector of the invention. “Pain gate” features of the injector physiologically block pain signals so that such pain signals are reduced and/or eliminated.

Accordingly, the injector reduces stress, fear, and/or anxiety experienced by the recipient of the injection, particularly those who have needle phobias.

Needle phobia is a common phenomenon that often results in decreased patient compliance with and patient adherence to medical care. The injector of the injection is likely to reduce induction of needle phobia, as compared with standard syringes and injectors, thereby improving life-long compliance with medical care. In particular, young recipients of injections using the injector of the invention are less likely to develop needle phobias, and thus are less likely to be reluctant to receive medical care throughout life.

For children who need to receive daily injections, use of the injectors of the invention can reduce conflict and struggle over administration of injections, thereby improving relationships between parents and children.

Further, use of the injectors of the invention may have beneficial effects on quality of life and/or treatment outcome, generally due to better patient compliance with and adherence to treatment via injections. For example, patients with existing needle phobias are less likely to be traumatized by the injectors of the invention.

Piercing the skin with a needle is a painful proposition in normal humans and animals. The needle is activating pain receptors in the skin, and this receptor activation is transmitted as a signal to the brain This pain signal transduction can be reduced by co-activation of mechanoreceptors in the skin. This concept is named the “Pain Gate” mechanism. While conventional standard syringes have no built-in features to activate the “pain gate” mechanism, the injectors of the invention can include such features. For example, the injectors of the invention can have a wide base, and/or can have protrusions from the base of the injector so as to activate the “pain gate” mechanism. The “pain gate” features of the injectors activate the “pain gate” before the needle of the injector pierces the skin, and can maintain activation of the “pain gate” throughout the injection.

Unlike known syringes and injectors, the injectors of the invention allows pre-selection of the injection site, and then rest on the injection site prior to injection, thereby reducing chances of target selection error.

Furthermore, the broad palm top of the injectors of the invention eliminates the need for the stabbing motion typically recommended when using known injectors and/or syringes. Consequently, because no stabbing movement is needed, the resulting injection is gentler and less menacing for individuals, particularly those with needle phobias.

The invention includes an embodiment that is a compact auto-injector device, having at least one concealed needle, the auto-injector device being shaped so as to appear friendly and non-threatening, and being adapted so as to encourage a gentle pressing action for triggering the device. Its friendly and non-threatening shape does not discourage its use. Moreover, the shape is conducive to proper application. It is intuitive to apply the device in the proper orientation, and the orientation does not need to change at any time during operation of the device. Since the shape of the device suggests application in the correct orientation, the possibility of shape-induced confusion regarding application orientation is significantly reduced.

The injectors of the invention can contain multiple doses of same medication or different medications.

The injectors of the invention can be convenient for carrying, including carrying in a small pocket, even when containing multiple doses of medication(s).

### BRIEF DESCRIPTION OF DRAWINGS

The invention will be more fully understood from the following detailed description, in conjunction with the following figures, wherein:

FIG. 1 is a cross-sectional view of an automatic injector having a syringe in side-by-side relationship with an injection/withdrawal mechanism.

FIG. 1A is a cross-sectional view of the automatic injector of FIG. 1 showing the safety pin removed, making the automatic injector ready for use.

FIG. 1B is a cross-sectional view of the automatic injector of FIG. 1A showing the trigger pushed in, and the injection/withdrawal mechanism activated.

FIG. 1C is a cross-sectional view of the automatic injector of FIG. 1B showing the injection needle inserted, and the injection progressing.

FIG. 1D is a cross-sectional view of the automatic injector of FIG. 1C after the injection has been completed, the withdrawal mechanism having just been activated.

FIG. 1E is a cross-sectional view of the automatic injector of FIG. 1D, showing the main spring collapsed, and the withdrawal spring ready to begin needle withdrawal.

FIG. 1F is a cross-sectional view of the automatic injector of FIG. 1E, showing the injection needle completely withdrawn and hidden inside the body.

FIG. 2 is a cross-sectional view of an automatic injector having a syringe in side-by-side relationship with an injection/withdrawal mechanism having a two-stage mode of operation to ensure complete insertion of an injection needle before injection.

FIG. 2A is a cross-sectional view of the automatic injector of FIG. 2, showing the safety pin removed, making the automatic injector ready for use.

FIG. 2B is a cross-sectional view of the automatic injector of FIG. 2A, showing the trigger pushed in and the injection/withdrawal mechanism activated.

FIG. 2C is a cross-sectional view of the automatic injector of FIG. 2B, showing the injection needle insertion into an injection site in progress.

FIG. 2D is a cross-sectional view of the automatic injector of FIG. 2C, showing the injection just as it is ready to begin as needle insertion progresses.

FIG. 2E is a cross-sectional view of the automatic injector of FIG. 2D, showing the injection in progress after needle insertion has been completed.

FIG. 2F is a cross-sectional view of the automatic injector of FIG. 2E, after the entire dose has been injected, showing the withdrawal mechanism activated.

FIG. 2G is a cross-sectional view of the automatic injector of FIG. 2F, showing the main spring collapsed, and the withdrawal spring ready to withdraw the needle.

FIG. 2H is a cross-sectional view of the automatic injector of FIG. 2G, showing the needle completely withdrawn and hidden inside body.

FIG. 3 is a cross-sectional view of a device for palm-controlled operation of a standard syringe, the palm receiving surface of the device being adapted to receive a palm in substantially parallel relationship to a surface of an injection site.

FIG. 3A is a cross-sectional view of the device of FIG. 3, showing the injection needle partially emerging from the device.

FIG. 3B is a cross-sectional view of the device of FIG. 3A, showing the injection needle completely extending out of the device, also showing injection about to begin.

FIG. 3C is a cross-sectional view of the device of FIG. 3B, showing the injection in progress.

FIG. 3D is a cross-sectional view of the device of FIG. 3C, showing the palm-controlled injection completed.

FIG. 3E is a cross-sectional view of the device of FIG. 3D, showing the device after withdrawal of the palm that operated the device.

FIG. 4 is a cross-sectional view of a device for palm-controlled operation of a standard syringe that automatically withdraws the injection needle.

FIG. 4A is a cross-sectional view of the device of FIG. 4, showing the injection needle partially emerging from the device.

FIG. 4B is a cross-sectional view of the device of FIG. 4A, showing the injection needle completely extended out of the device, and showing the injection ready to begin.

FIG. 4C is a cross-sectional view of the device of FIG. 4B, showing the injection in progress.

FIG. 4D is a cross-sectional view of the device of FIG. 4C, showing the palm-controlled injection completed.

FIG. 4E is a cross-sectional view of the device of FIG. 4D, showing the retrieval of the syringe about to begin.

FIG. 4F is a cross-sectional view of the device of FIG. 4E, showing the device after withdrawal of the palm that operated the device, and showing the injection needle being completely withdrawn.

FIG. 5 is a cross-sectional view of a device for simultaneous palm-controlled operation of multiple standard syringes that automatically withdraws the corresponding injection needles.

FIG. 5A is a cross-sectional view of the device of FIG. 5, showing the palm-controlled injections completed.

FIG. 6 is a cross-sectional view of a device for simultaneous palm-controlled operation of multiple standard syringes.

FIG. 7 is a cross-sectional view of an automatic injector having a syringe in coaxial relationship with an injection/withdrawal mechanism.

FIG. 7A is a cross-sectional view of the automatic injector of FIG. 7, showing the safety pin removed, making the automatic injector ready for use.

FIG. 7B is a cross-sectional view of the automatic injector of FIG. 7A, showing the top trigger pushed in, and the injection/withdrawal mechanism activated.

FIG. 7C is a cross-sectional view of the automatic injector of FIG. 7B, showing the injection needle inserted, and the injection in progress.

FIG. 7D is a cross-sectional view of the automatic injector of FIG. 7C, showing the device after the injection has been completed, and showing the withdrawal mechanism activated.

FIG. 7E is a cross-sectional view of the automatic injector of FIG. 7D, showing the main spring operation reversed, and showing the start of needle withdrawal.

FIG. 7F is a cross-sectional view of the automatic injector of FIG. 7E, showing the injection needle completely withdrawn.

FIG. 8 is a cross-sectional view of a single automatic injector having a syringe in side-by-side relationship with an injection/withdrawal mechanism, the automatic injector to be used in a multi-dose automatic injector.

FIG. 8A is a top view of the single automatic injector of FIG. 8.

FIG. 9 is a top view of a multi-dose automatic injector having four automatic injectors, each as shown in FIGS. 8 and 8A.

FIG. 9A is a bottom view of the multi-dose automatic injector of FIG. 9, showing the safety pins in overlapping relationship.

FIG. 10 is a side view of an automatic palm activated injector, showing use of the automatic palm activated injector by application to a person's thigh.

FIG. 10A is a side view of an automatic palm activated injector, showing use of the automatic palm activated injector by application to a person's thigh, the injection surface of the automatic palm activated injector being concave so as to substantially fit the convex curvature of the person's thigh.

FIG. 11 is a line drawing depicting an automatic palm activated injector being applied to a thigh of a person.

FIG. 12 is a cross-sectional view of a device for palm-controlled operation of a standard syringe, the palm receiving surface of the device being adapted to receive a palm in substantially perpendicular relationship to a surface of an injection site.

FIG. 13 is a line drawing depicting an automatic palm activated injector being applied to a thigh of a person with a palm in substantially perpendicular relationship to a surface of an injection site on the thigh.

### DETAILED DESCRIPTION

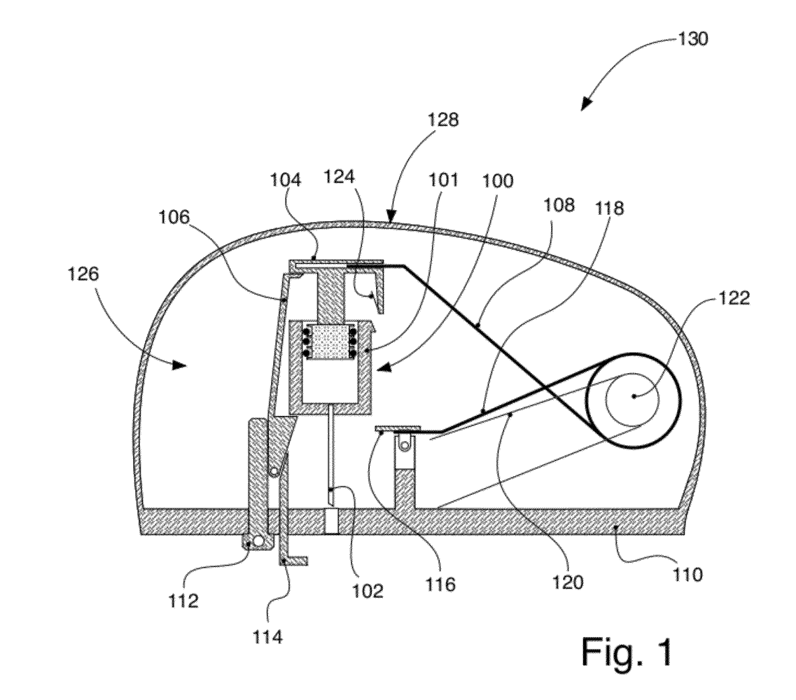

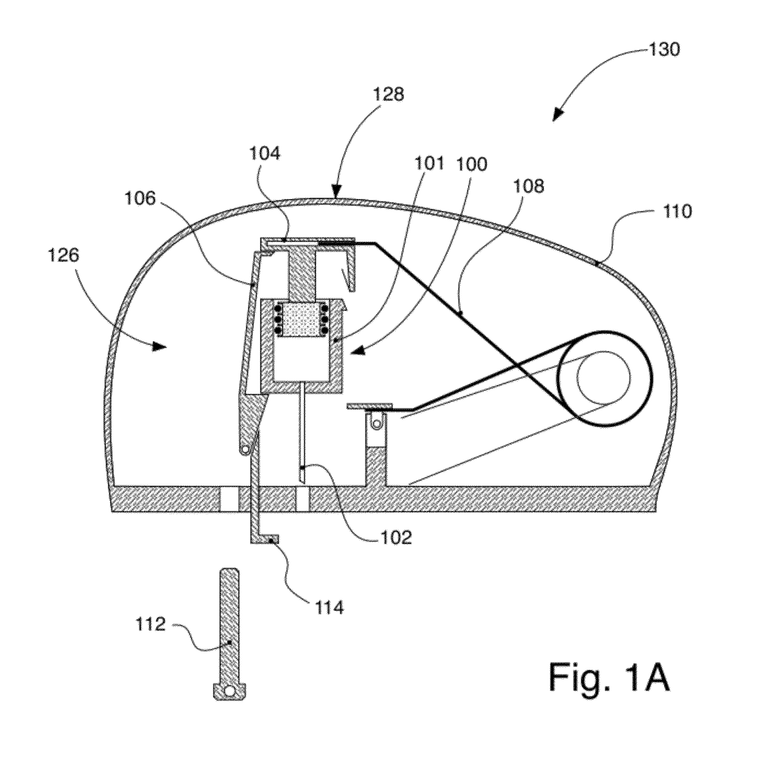

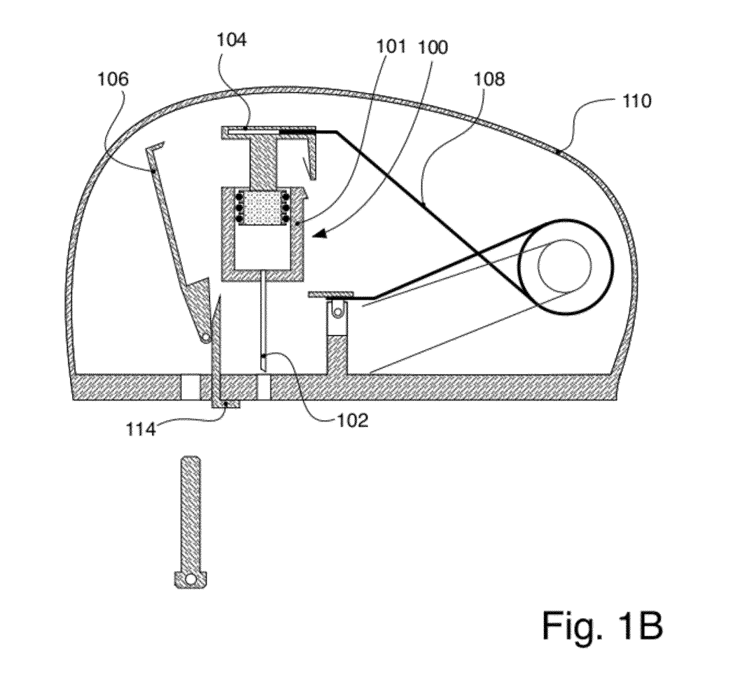

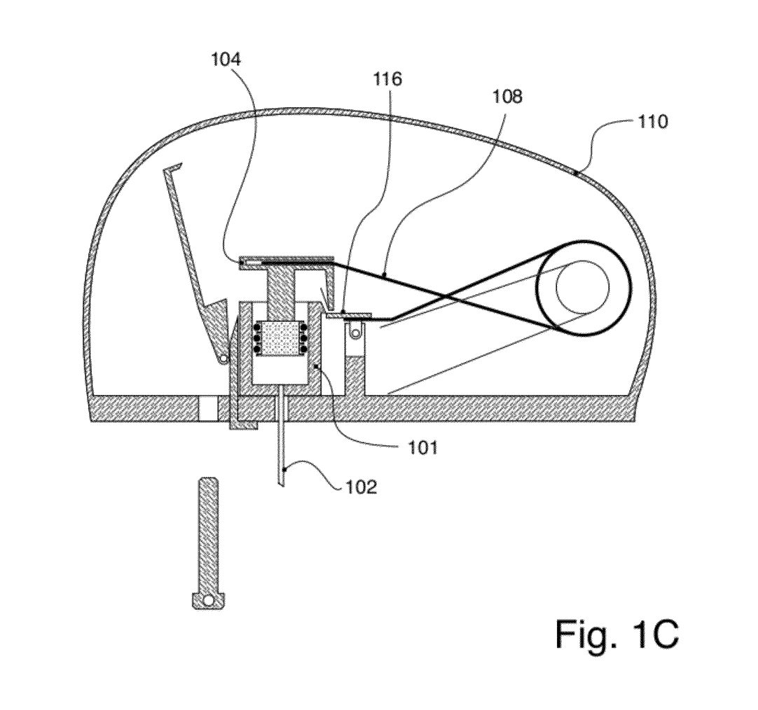

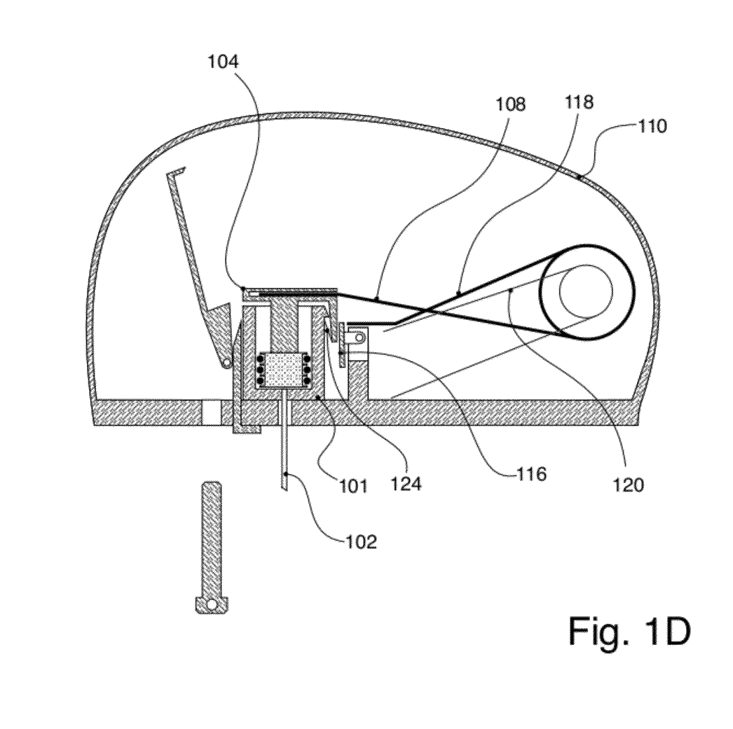

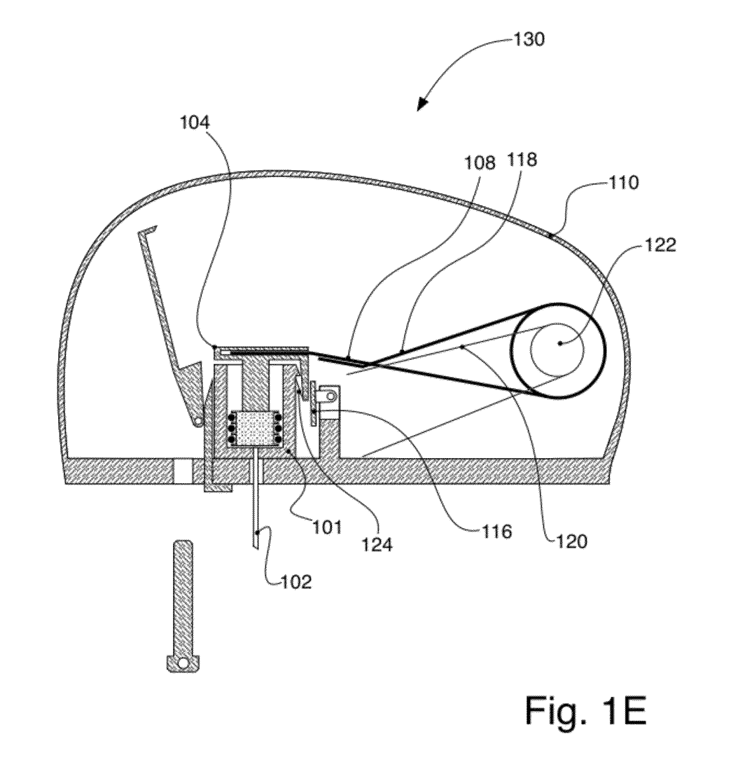

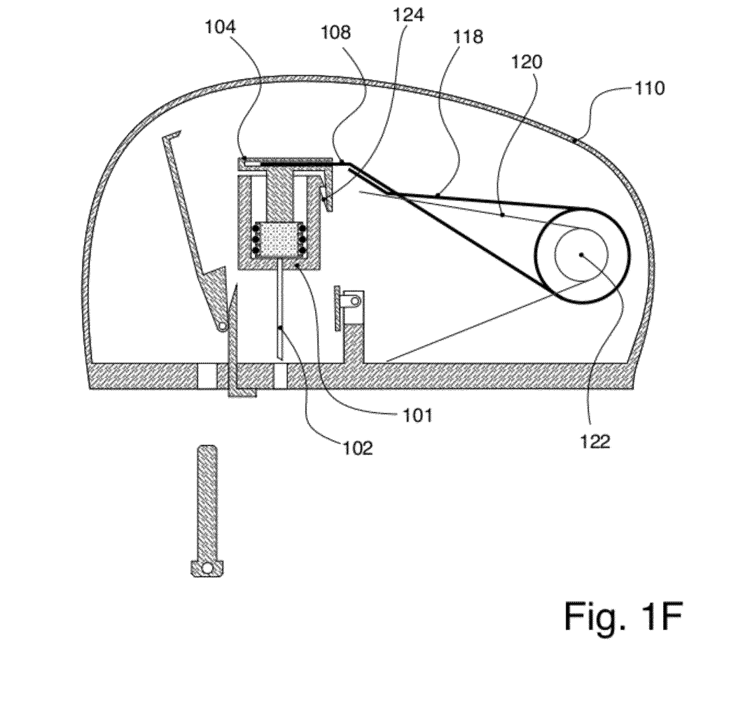

With reference to FIG. 1, a syringe 100 has a cylinder 101 containing a substance to be injected, and has a needle 102 and a plunger 104. A stopper 106 prevents a top arm 108 of a main spring from pushing the plunger 104 into the cylinder 101. Stopper 106 also prevents syringe 100 from accidentally moving down and exposing the needle 102 through an opening in body 110. A safety pin 112 prevents a trigger 114 from initiating an injection sequence. To initiate an injection sequence, the safety pin 112 is removed, and the trigger 114 is pushed in. Pushing the trigger 114 in causes the stopper 106 to swing out, thereby enabling the top arm 108 of the main spring to push the plunger 104 downward. When plunger 104 reaches a retrieval trigger 116, a bottom arm 118 of main spring will be free to move up and enable a retrieval spring 120 to rotate around an axle 122. An interlocking spring 124 will interlock plunger 104 with cylinder 101, when plunger 104 is fully inserted in cylinder 101. All of the parts contained within the body 110 are referred to as an injector 126, which includes the syringe 100, and other parts described above that cooperate so as to perform an injection using the syringe 100. The body 110 has a palm-receiving surface 128 that receives a palm of a hand. The palm-receiving surface 128 is cooperative with the injector 126 to as to provide a palm-controlled device 130 for injection of a substance into an injection site of an organism. The palm-receiving surface 128 is shaped to receive a palm of a hand so that when the palm of the hand that is used to operate the palm-controlled device 130, the palm must be substantially parallel to a surface of the injection site. When pressure is applied to the palm-receiving surface 128, the injector 126 is actuated so as to inject the contents of at least one pre-filled syringe of the injector into the injection site. The organism can be a human or an animal.

With reference to FIG. 1A, prior to removal of safety pin 112, the trigger 114 cannot be pushed in when pressure is applied to the palm-receiving surface 128. Consequently, the injector 126 cannot be actuated, and no injection can occur. When the safety pin 112 is removed from the body 110, the device 130 is ready for use. Stopper 106 still prevents the top arm 108 of the main spring from pushing down the plunger 104. The stopper 106 also holds the cylinder 101 from accidentally moving down, thereby exposing the injection needle 102.

With reference to FIG. 1B, body 110 is pressed against the intended injection site of an organism, thereby pushing trigger 114 into body 110. Trigger 114 pushes stopper 106 out of the way, thereby enabling the top arm 108 of the main spring to push the plunger 104 downward. Due to natural viscosity and lack of compressibility of the liquid substance in the cylinder 101, pressing on the plunger 104 causes the cylinder 101 to move downward, along with the needle 102, thereby causing the needle 102 to move through the opening in body 110. Once the needle emerges from the hole in the body 110, it begins to enter the injection site of the organism.

With reference to FIG. 1C, when cylinder 101 contacts the body 110, the needle 102 has completely emerged from the body 110. Then, the top arm 108 of the main spring continues to push the plunger 104 into the cylinder 101, causing injection of the substance through the needle 102 until the plunger 104 activates the withdrawal trigger 116.

With reference to FIG. 1D, top arm 108 of the main spring continues to push plunger 104 to cause the interlocking spring 124 of the plunger 104 to latch onto the cylinder 101. The top arm 108 of the main spring continues to push, causing injection of all of the substance. When both the plunger 104 and the cylinder 101 have each reached the full travel, the plunger 104 activates withdrawal trigger 116. Activation of withdrawal trigger 116 releases bottom arm 118 of the main spring. Withdrawal spring 120 rests on the bottom arm 118 of the main spring. The withdrawal spring 120 is now free to push both the bottom arm 118 and the top arm 108 of the main spring upward.

With reference to FIG. 1E, the bottom arm 118 of the main spring moves up and rests against the top arm 108 of the main spring. Withdrawal spring 120 rests on the bottom arm 118 of the main spring. The withdrawal spring 120 is now free to push the bottom arm 118 of the main spring against the top arm 108 of the main spring, to cause the main spring to rotate around axle 122, which will lift the plunger 104. Since the interlocking spring 124 of the plunger 104 is in latched relationship with the cylinder 101, the cylinder 101 will be lifted along with the plunger 104. Lifting the cylinder 101 will lift the injection needle 102 upwards, withdrawing the injection needle 102 back into the body 110 of the palm controlled device 130.

With reference to FIG. 1F, withdrawal spring 120 rotates both bottom arm 118 of the main spring and top arm 108 of the main spring around the axle 122. Rotation of the top arm 108 of the main spring pulls plunger 104 upward. Since the interlocking spring 124 of the plunger 104 is in latched relationship with the cylinder 101, the cylinder 101 is lifted along with the plunger 104. Lifting the cylinder 101 lifts the injection needle 102 upwards, thereby withdrawing the injection needle 102 back into the body 110 of the palm controlled device 130.

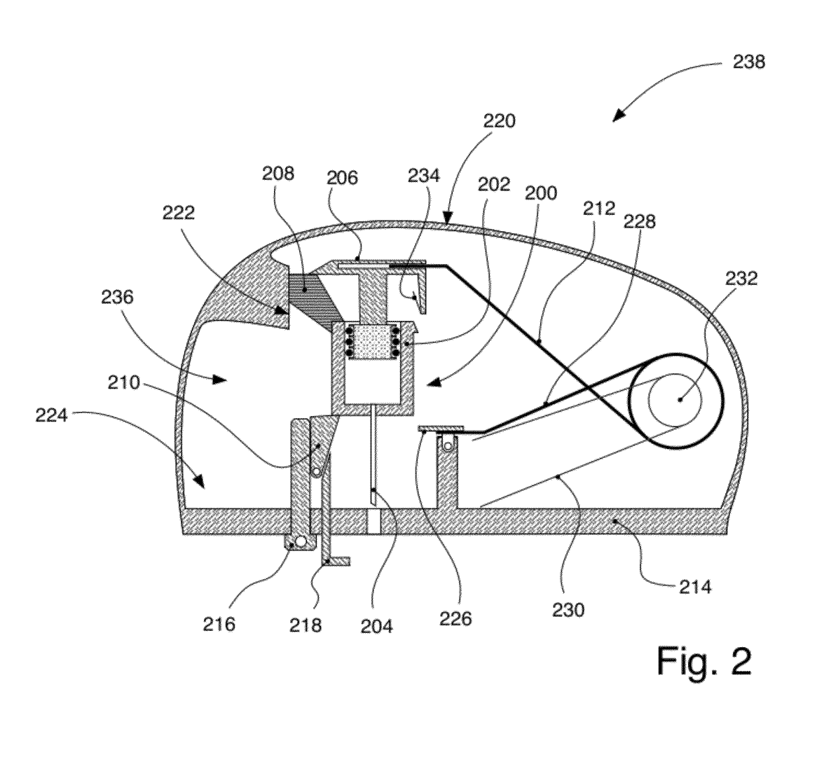

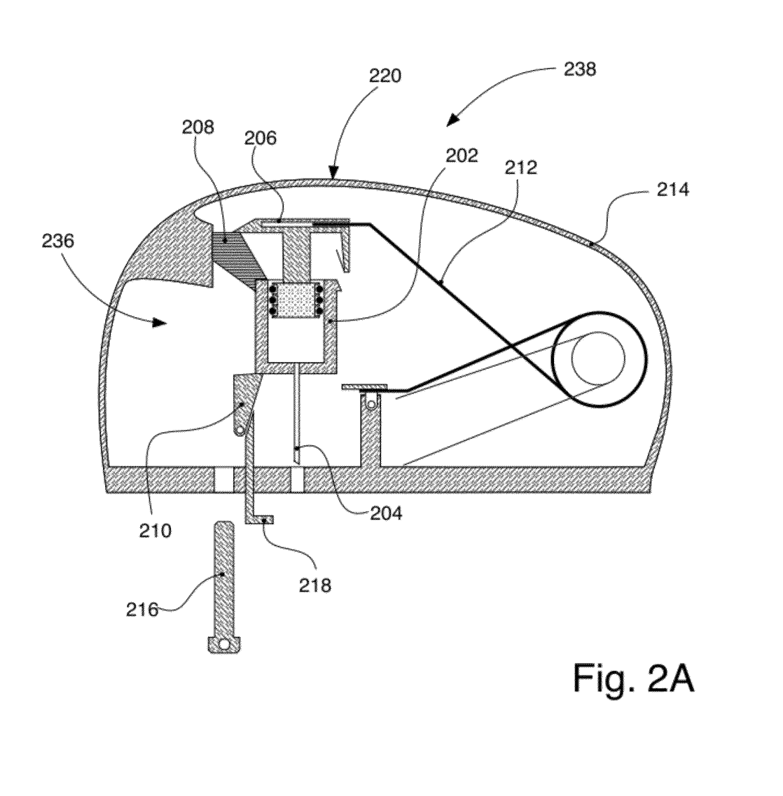

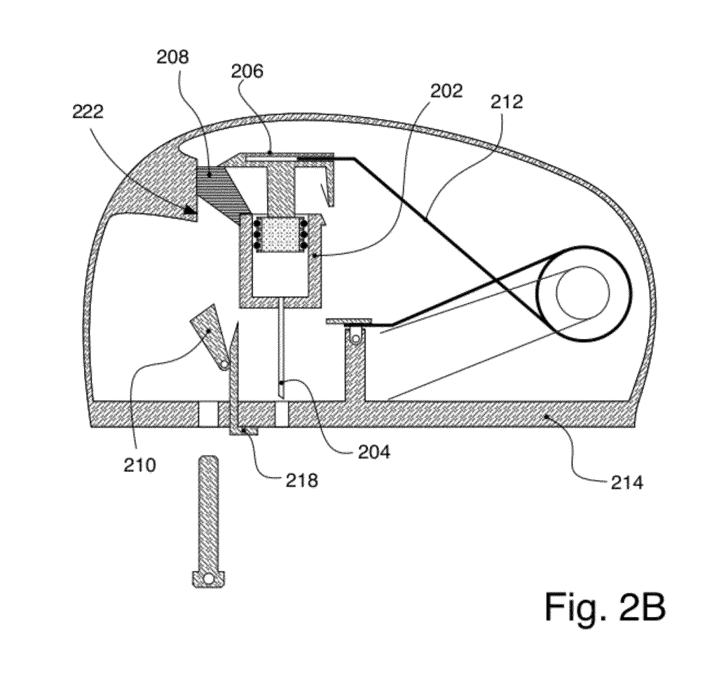

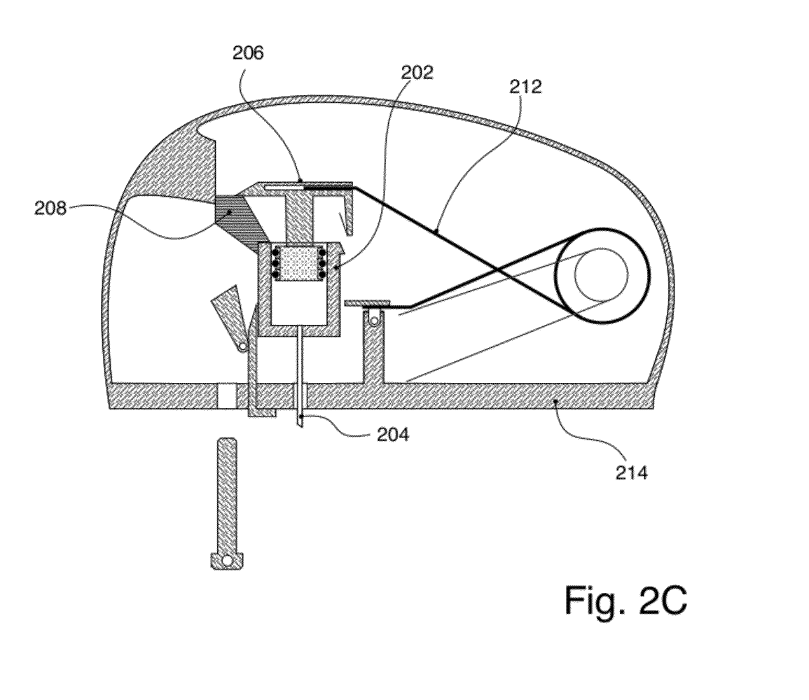

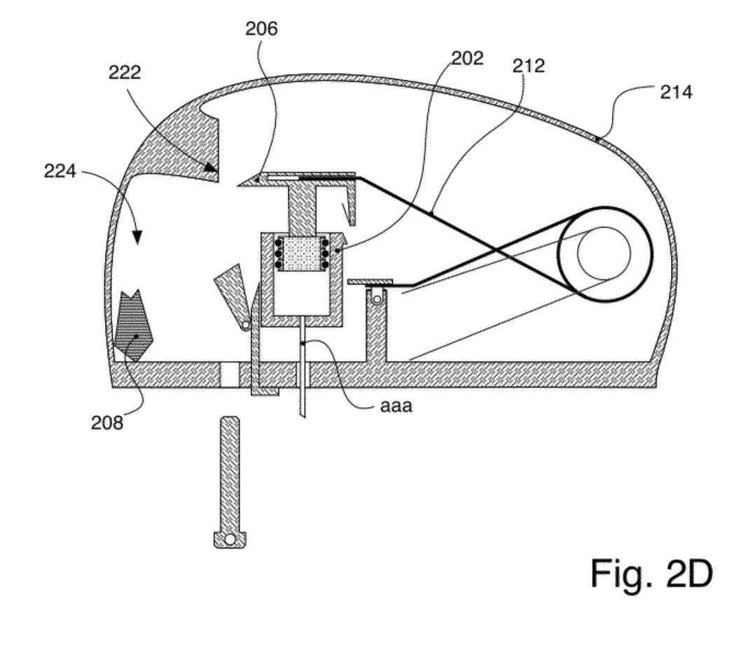

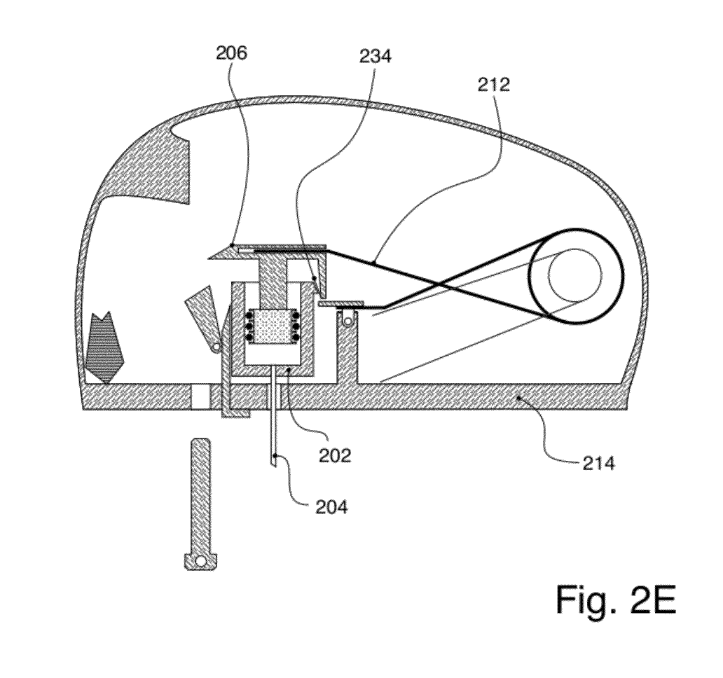

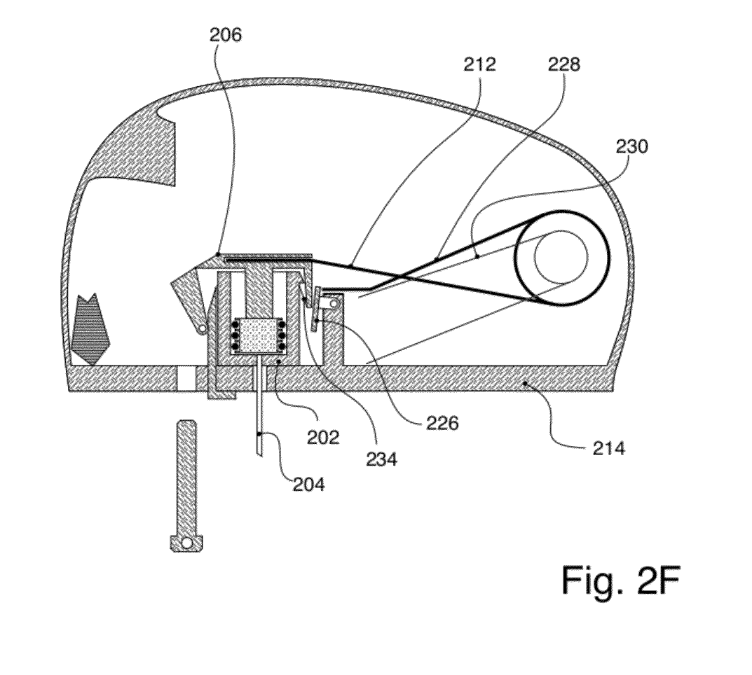

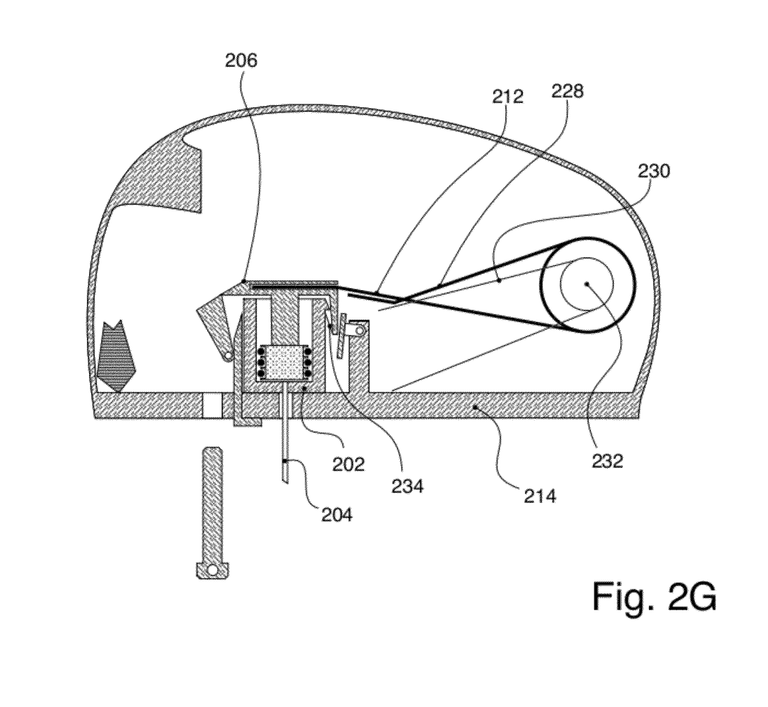

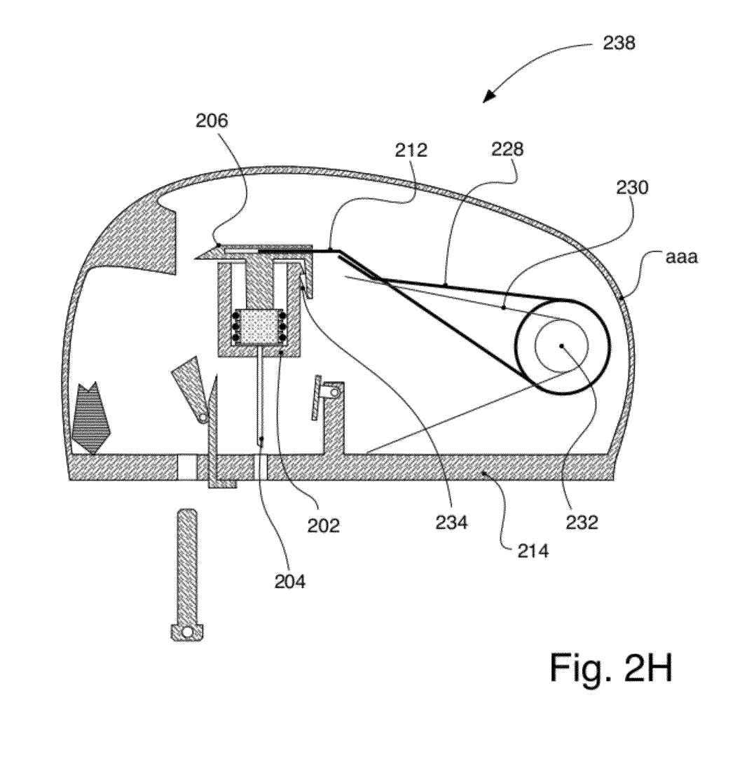

With reference to FIG. 2, a syringe 200 has a cylinder 202 containing a substance to be injected, and has an injection needle 204 and a plunger 206. Retainer 208 prevents relative movement between plunger 206 and the cylinder 202. A stopper 210 prevents a top arm 212 of a main spring from pushing the plunger 206, the retainer 208, the cylinder 202, and the injection needle 204 downward. Stopper 210 prevents syringe 200 from accidentally moving down, thereby preventing exposure of the needle 204 through an opening in body 214, so as to prevent needle stick accidents. A safety pin 216 prevents a trigger 218 from initiating an injection sequence. To initiate an injection sequence, the safety pin 216 is removed, and pressure applied by the palm of a hand onto the palm-receiving surface 220 of the body 214 causes body 214 to press against the injection area containing the intended injection site of the organism. The counter-pressure of the injection area pushes the trigger 218 inward. Pushing the trigger 218 inward causes the stopper 210 to swing out, thereby enabling the top arm 212 of the main spring to push the plunger 206 downward. Retainer 208 prevents the plunger 206 from entering the cylinder 202. Retainer 208 is free to glide along straight edge 222 of the body 214 until the retainer 208 is pushed into an open area 224, thereby permitting the plunger 206 to move further into the cylinder 202. When plunger 206 reaches a retrieval trigger 226, a bottom arm 228 of the main spring will be free to move up and enable a retrieval spring 230 to rotate about an axle 232. An interlocking spring 234 will interlock the plunger 206 with the cylinder 202, when plunger 206 is fully inserted in cylinder 202. All of the parts contained within the body 214 are referred to as an injector 236, which includes the syringe 200, and other parts described above that cooperate so as to perform an injection using the syringe 200. The body 214 has a palm-receiving surface 220 that receives a palm of a hand. The palm-receiving surface 220 is cooperative with the injector 236 so as to provide a palm-controlled device 238 for injection of a substance into an injection site of an organism. The palm-receiving surface 220 is shaped to receive a palm of a hand so that when the palm of the hand that is used to operate the palm-controlled device 238, the palm must be substantially parallel to a surface of the injection site. When pressure is applied to the palm-receiving surface 220, the injector 236 is actuated so as to inject the contents of at least one pre-filled syringe 200 of the injector 236 into the injection site. The organism can be a human or an animal.

With reference to FIG. 2A, prior to removal of safety pin 216, the trigger 218 cannot be pushed in when pressure is applied to the palm-receiving surface 220. Consequently, the injector 236 cannot be actuated, and no injection can occur. When the safety pin 216 is removed from the body 214, the device 238 is ready for use. Stopper 210 still prevents the top arm 212 of the main spring from pushing down the plunger 206, the retainer 208, and the cylinder 202, thereby pushing the injection needle 204 out of the body 214.

With reference to FIG. 2B, the body 214 is pressed against the intended injection area, thereby moving the trigger 218 into the body 214. Trigger 218 pushes the stopper 210 out of the way, and enables the top arm 212 of the main spring to push the plunger 206. Retainer 208 can glide vertically alongside a straight edge 222 of the body 214, thereby forcing the cylinder 202 to move down together with the plunger 206 to begin insertion of the needle 204 into the organism.

With reference to FIG. 2C, the top arm 212 of the main spring pushes the plunger 206 down. The retainer 208 prevents relative motion between the plunger 206 and cylinder 202, thereby pushing the cylinder 202 down. The needle 204 protrudes from the body 214 and into the organism.

With reference to FIG. 2D, top arm 212 of the main spring pushes the plunger 206 and the retainer 208 down until the retainer 208 is pushed past the straight edge 222 and into the open area 224 in the body 214. The retainer 208, having been pushed out of place, no longer prevents relative movement between the plunger 206 and the cylinder 202.

With reference to FIG. 2E, the top arm 212 of the main spring continues to push the plunger 206. While injecting, the natural viscosity and incompressibility of the fluid contained in the cylinder 202, together with the small resistance of the interlocking spring 234 continue to push the cylinder 202 and the injection needle 204 out of the body 214.

With reference to FIG. 2F, top arm 212 of the main spring continues to push the plunger 206, and the plunger 206 continues to push the cylinder 202 down so that the interlocking spring 234 of the plunger 206 latches onto the cylinder 202. Top arm 212 of the main spring continues to push down on the plunger 206 until the plunger 206 is fully inserted into the cylinder 202, and the injection needle 204 has completely emerged from the body 214. The plunger 204 activates withdrawal trigger 226. Activation of the withdrawal trigger 226 releases the bottom arm 228 of the main spring. The withdrawal spring 230 rests on the bottom arm 228 of the main spring, and the withdrawal spring 230 is now free to push up the bottom arm 228 of the main spring.

With reference to FIG. 2G, the withdrawal spring 230 pushes the bottom arm 228 of the main spring up against the top arm 212 of the main spring, thereby causing the main spring to rotate about the axle 232. The top arm 212 of the main spring pulls the plunger 206 upward. The plunger 206 is interlocked with the cylinder 202 because of the interlocking spring 234, thereby pulling the cylinder 202 upward. Pulling the cylinder 202 upward causes withdrawal of the injection needle 204 into the body 214.

With reference to FIG. 2H, the withdrawal spring 230 rotates both the bottom arm 228 of the main spring and the top arm 212 of the main spring about the axle 232. Rotation of the top arm 212 of the main spring pulls the plunger 206 upward. The interlocking spring 234 latches plunger 206 to the cylinder 202. Pulling the plunger 206 upward also pulls the cylinder 202 upward. Since the injection needle 204 is connected to the cylinder 202, the injection needle 204 is withdrawn completely into the body 214 of the device 238.

### Single-Dose Palm-Controlled Injector

Daily home-based administration of medications has gained widespread use, including growth hormones, insulin, heparin, antibiotics, IVF hormones, for example. Caregivers and patients are often intimidated by the stab-like motion of the injection, and the pain inflicted thereby. Consequently, there is reluctance and commotion associated with administration of injections using known injectors in many households.

The Single-dose Palm-controlled Injector of the invention employs a palm-controlled method of injection, as well as “pain gate” activation features, to provide a more comfortable experience of needed injections.

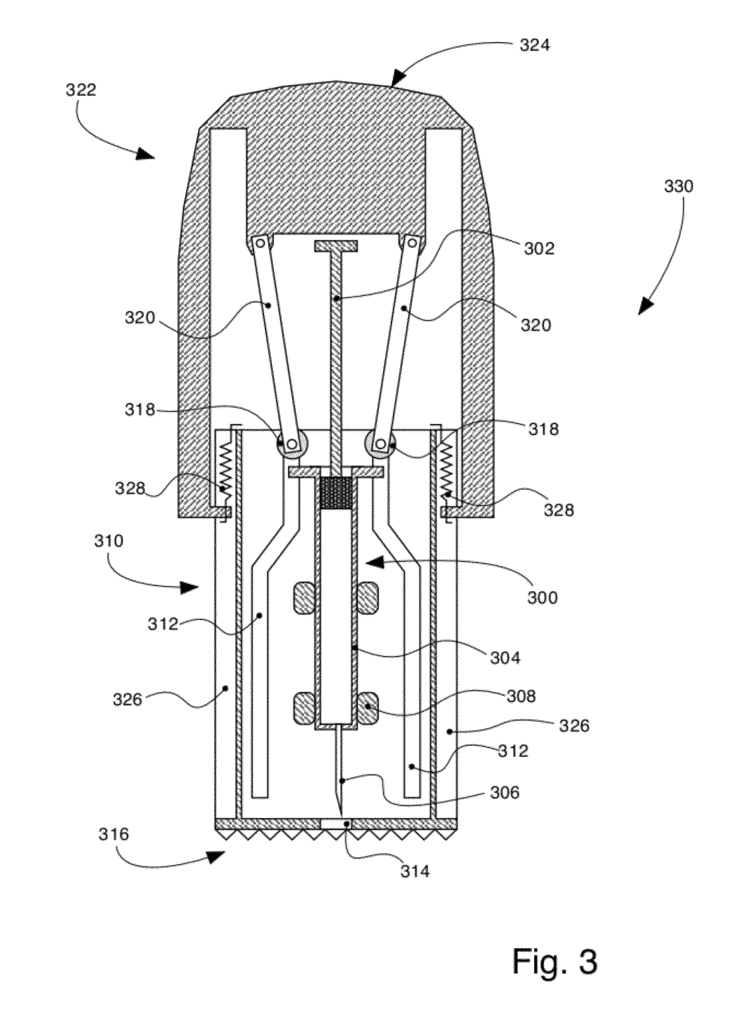

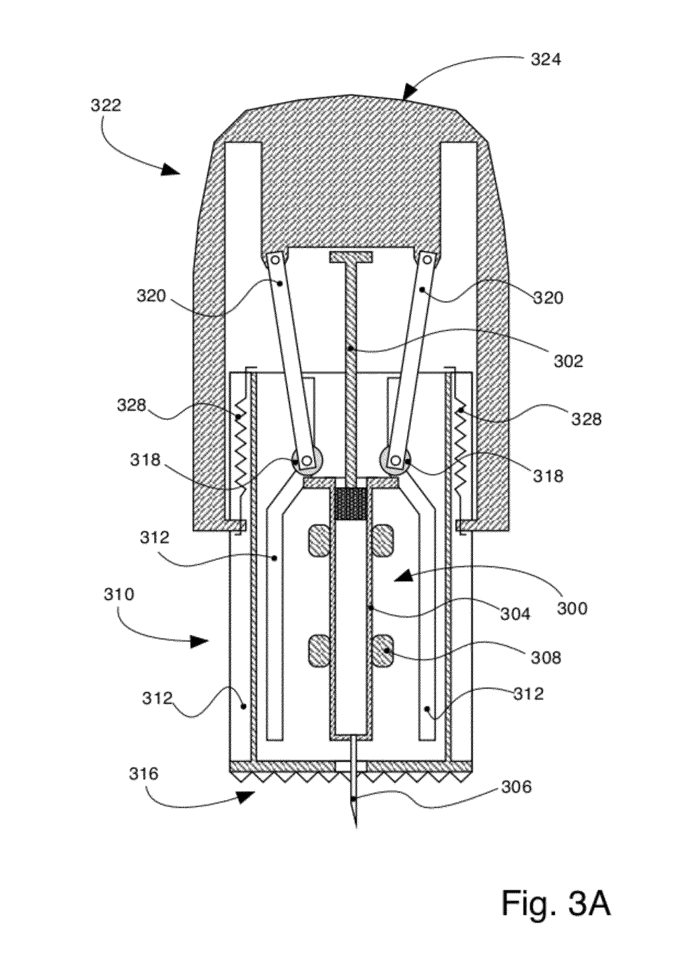

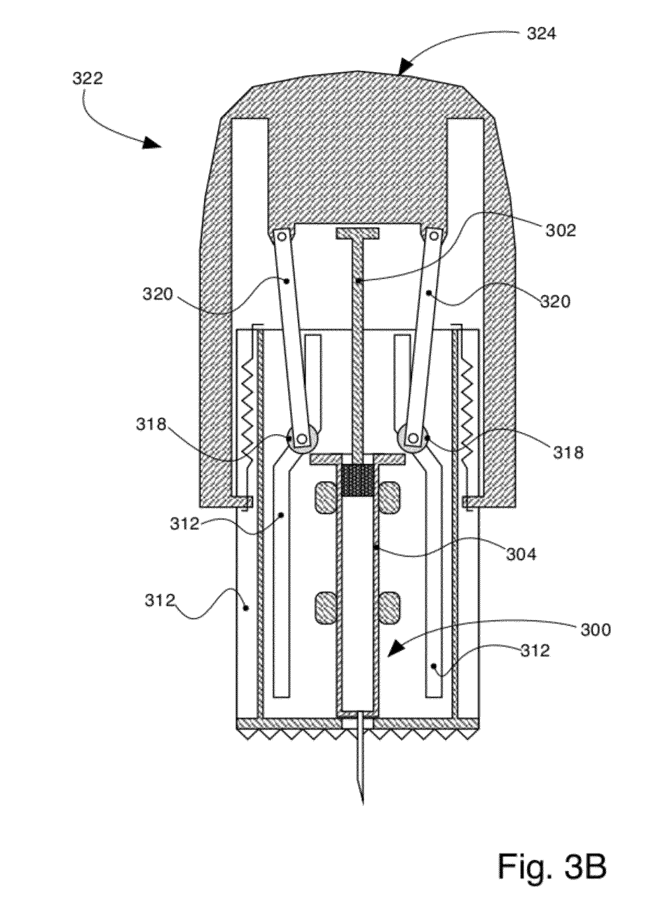

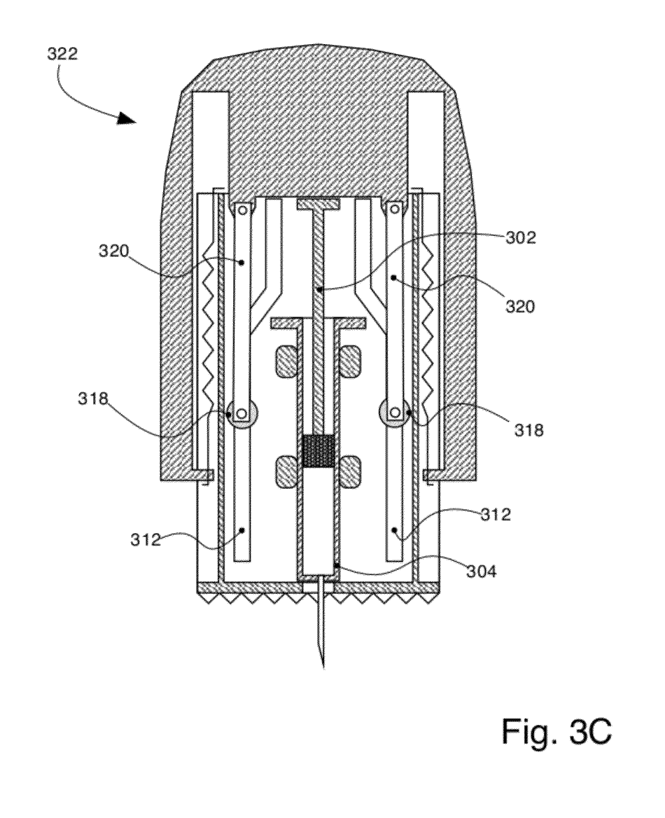

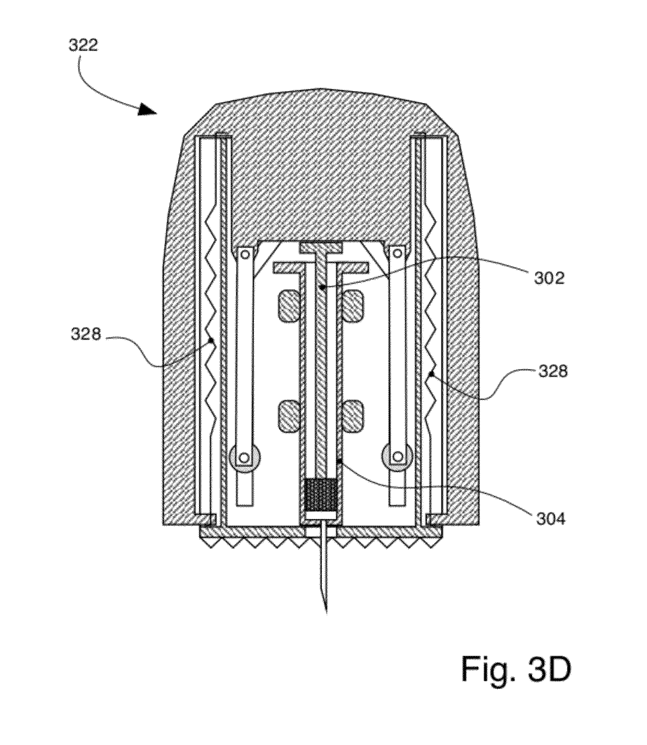

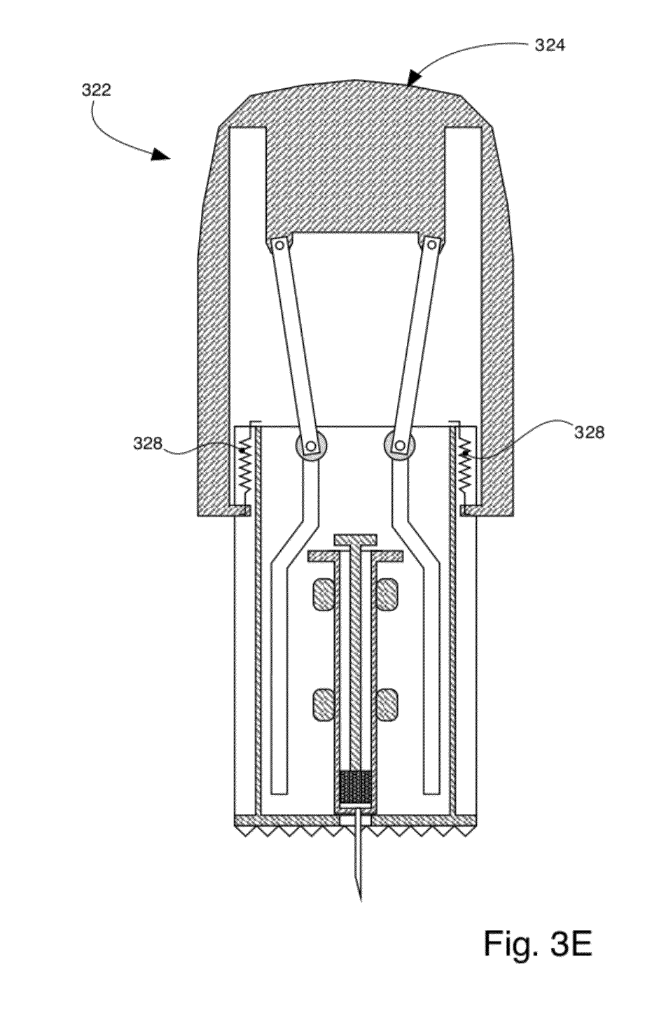

With reference to FIG. 3, a syringe 300, having a plunger 302, a cylinder 304, and an injection needle 306, is releasably and slidably held by syringe holders 308. The syringe holders 308 are attached to the base 310. The base 310 includes guide tracks 312. The bottom of the base 310 contacts an injection area of the organism to be injected, and the injection site falls within the injection area. The bottom of the base 310 includes a hole 314 which allows the injection needle 306 to pass through. The bottom of the base 310 also can include at least one pain gate feature 316, such as a substantially flat surface with gently rounded edges, or a plurality of bumps, or a plurality of ridges, such as concentric ridges, or straight ridges, or S-shaped ridges, or L-shaped ridges, or radial ridges. The guide tracks 312 constrain movement of rollers 318, each roller 318 being rotatably attached to a respective arm 320. Each arm 320 is hingedly attached to a top 322, the top 322 having a palm-receiving surface 324. The palm-receiving surface 324 receives pressure as applied by a palm of a hand, the palm-receiving surface 324 being shaped so that the palm is substantially parallel to a surface of an injection site of an organism while operating the device. The top 322 is in slidable relationship with the base 310, the top 322 being movable along spring tracks 326. The springs 328 apply a restoring force between the top 322 and the base 310 when the top 322 is pressed by a palm towards the base 310.

All of the parts 308-320 and 326-328 are referred to as an injector, which parts cooperate so as to perform an injection using the syringe 300. The top 322 has a palm-receiving surface 324 that receives a palm of a hand. The palm-receiving surface 324 is cooperative with the injector to as to provide a palm-controlled device 330 for injection of a substance into an injection site of an organism.

With reference to FIG. 3A, a palm of a hand presses onto the palm-receiving surface 324 of the top 322, thereby applying pressure to the injection area having the injection site, the pressure being applied via the pain gate features 316 of the base 310. The pressure also pushes arms 320 downward, thereby causing the arms 320 with rollers 318 to glide along path 312, the rollers 318 pushing the cylinder 304, thereby causing the syringe 300 to slide through the syringe holders 308, and causing the injection needle 306 to emerge from the hole 314. The top 322 does not touch the plunger 302, and so the injection needle 306 is being inserted into the injection site, without injecting the substance. The movement of the top 322 relative to the base 310 is resisted by the springs 328, causing the springs to be further stretched, thereby accumulating potential energy that will restore the top to its original position when the palm is removed.

With reference to FIG. 3B, the palm continues to press on the palm receiving surface 324 of the top 322, thereby moving the cylinder 304 until it reaches the end of its travel caused by the arms 320. The travel caused by the arms 320 ends when the rollers 318 spread wider than the width of the top end of the cylinder 304. The rollers 318 are led by the arms 320, the arms 320 being led by the path 312. Further, because the rollers have lost contact with the cylinder 304, further pushing of the top 322 will result in an inner surface of the top 322 pushing the plunger 302 into the cylinder 304.

With reference to FIG. 3C, the end of each arm 320 is guided by the paths 312, causing the rollers 318 to no longer contact the top the cylinder 304, while the inner surface of the top 322 pushes the plunger 302 into the cylinder 304, thereby causing injection of the substance into the injection site.

With reference to FIG. 3D, the top 322 has reached the lowest point in its travel, and the plunger 302 has reached the end of its travel within the cylinder 304, and the springs 328 have reached their maximum extension. As a result of the plunger 302 reaching the end of it's travel within the cylinder 304, the injection of the substance is completed.

With reference to FIG. 3E, when the pressure of the palm upon the palm receiving surface 324 of the top 322 is removed, the springs 328 are allowed to return their initial pre-loaded state. The contraction of the springs 328 drives the top 322 to return to its initial position. In this embodiment, we recommend using a syringe that automatically withdraws the injection needle into the syringe after injection of the substance is completed.

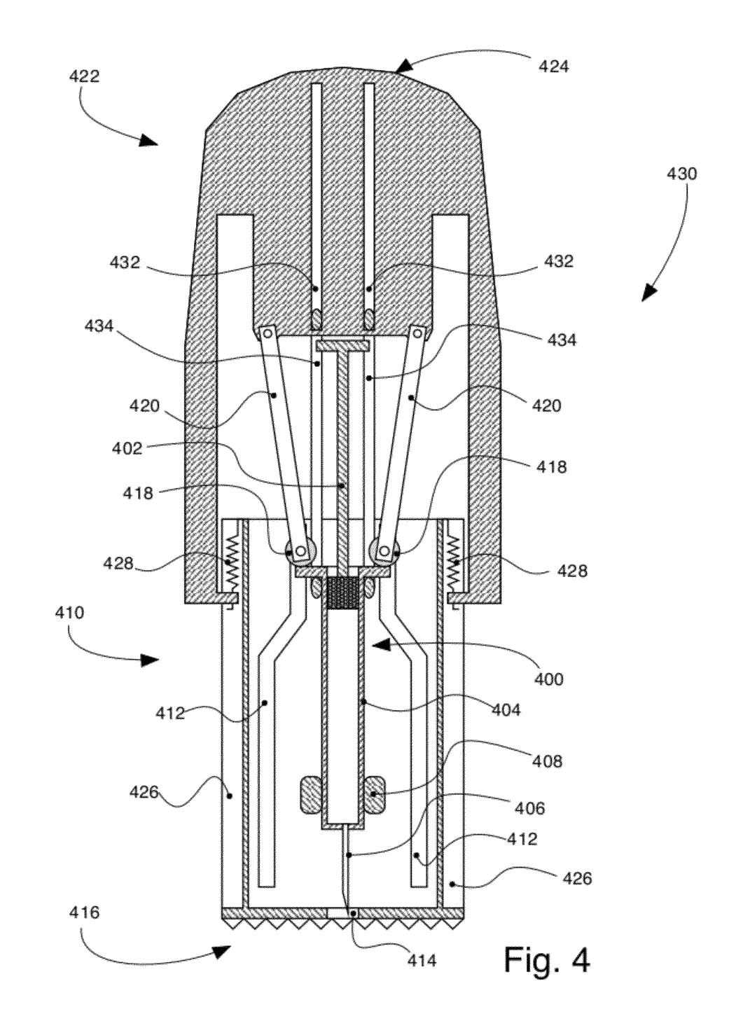

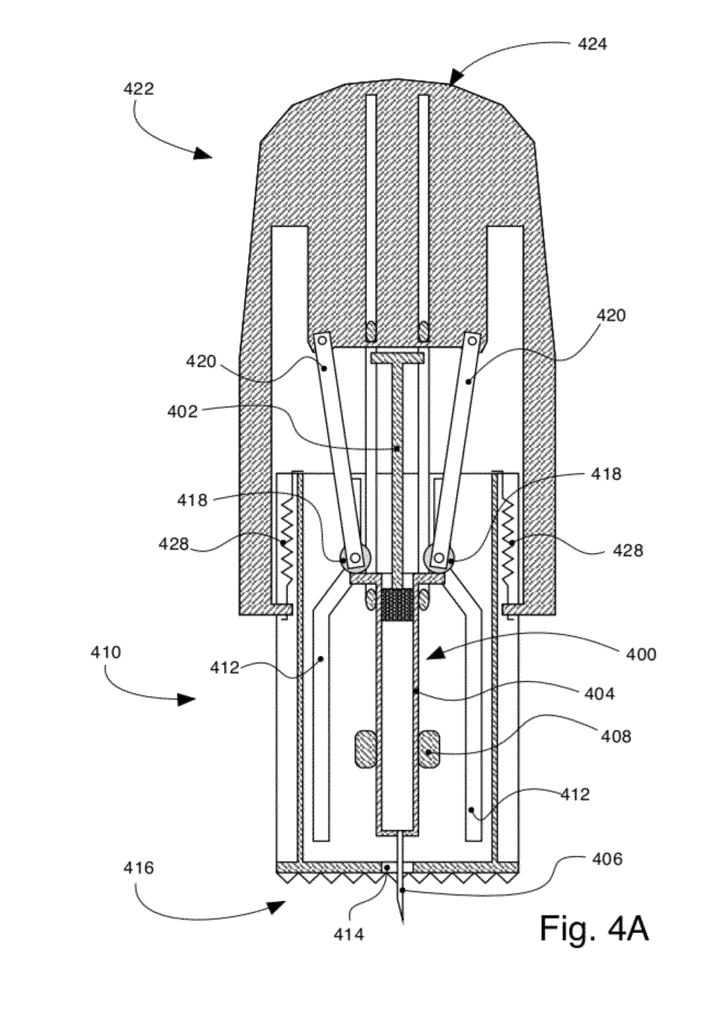

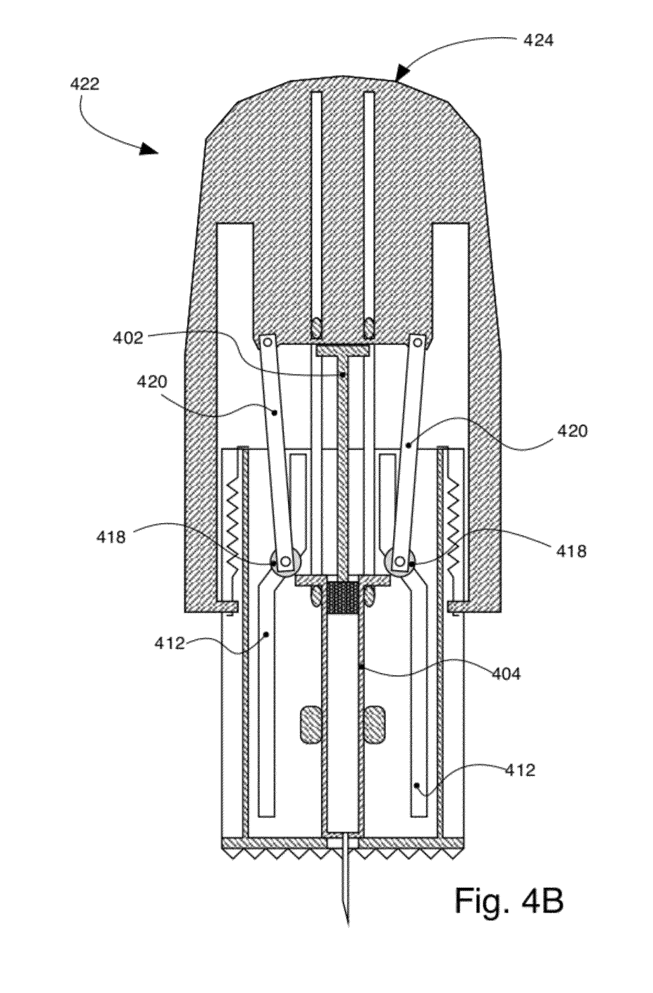

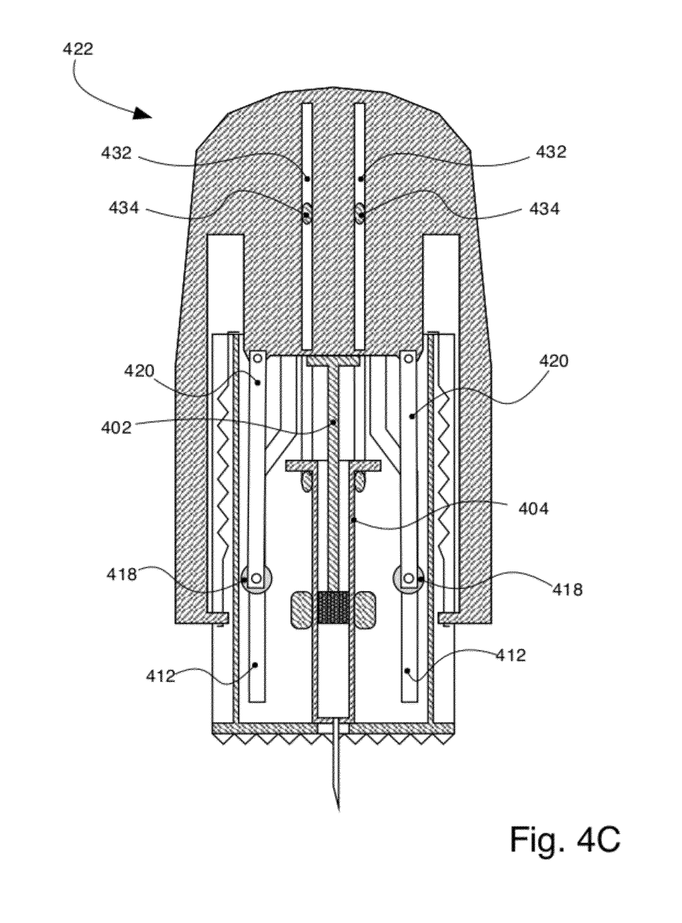

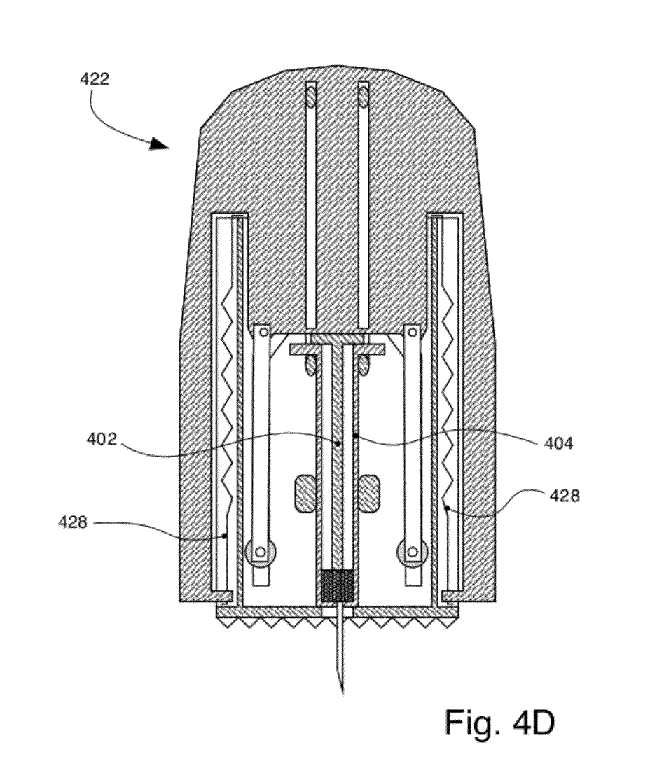

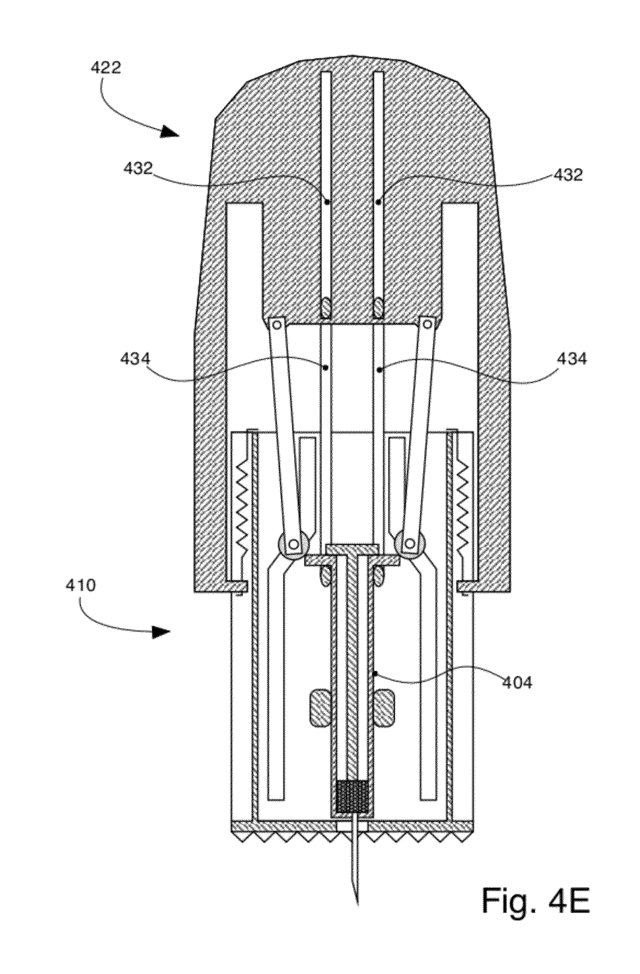

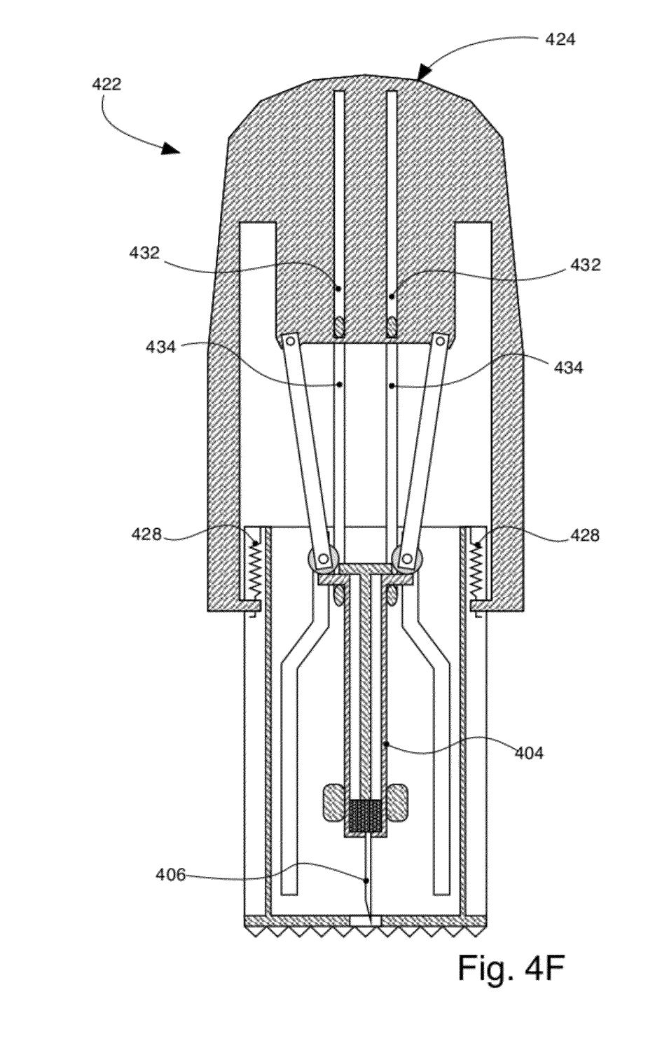

With reference to FIG. 4, a syringe 400, having a plunger 402, a cylinder 404, and an injection needle 406, is releasably and slidably held by syringe holders 408. The syringe holders 408 are attached to the base 410. The base 410 includes guide tracks 412. The bottom of the base 410 contacts an injection area of the organism to be injected, and the injection site falls within the injection area. The bottom of the base 410 includes a hole 414 which allows the injection needle 406 to pass through. The bottom of the base 410 also can include at least one pain gate feature 416, such as a substantially flat surface with gently rounded edges, or a plurality of bumps, or a plurality of ridges, such as concentric ridges, or straight ridges, or S-shaped ridges, or L-shaped ridges, or radial ridges. The guide tracks 412 constrain movement of rollers 418, each roller 418 being rotatably attached to a respective arm 420. Each arm 420 is hingedly attached to a top 422, the top 422 having a palm-receiving surface 424. The palm-receiving surface 424 receives pressure as applied by a palm of a hand, the palm-receiving surface 424 being shaped so that the palm is substantially parallel to a surface of an injection site of an organism while operating the device. The top 422 is in slidable relationship with the base 410, the top 422 being movable along spring tracks 426. The springs 428 apply a restoring force between the top 422 and the base 410 when the top 422 is pressed by a palm towards the base 410.

All of the parts 408-420 and 426-428 are referred to as an injector, which parts cooperate so as to perform an injection using the syringe 400. The top 422 has a palm-receiving surface 424 that receives a palm of a hand, cutouts 432, and syringe retrievers 434 that are free to move along the cutouts 432. The syringe retrievers 434 retrieve the cylinder 404 as the top 422 returns to its initial position. The palm-receiving surface 424 is cooperative with the injector to as to provide a palm-controlled device 430 for injection of a substance into an injection site of an organism. While top 422 returns to its initial position, the syringe retrievers 434 reach the end of the cutouts 432 in the top 422, the syringe retrievers 434 thereby beginning retrieving the cylinder 404. As the syringe retrievers 434 move, they pull the cylinder 404, thereby pulling the needle 406 into the base 410.

With reference to FIG. 4A, a palm of a hand presses onto the palm-receiving surface 424 of the top 422, thereby applying pressure to the injection area having the injection site, the pressure being applied via the pain gate features 416 of the base 410. The pressure also pushes arms 420 downward, thereby causing the arms 420 with rollers 418 to glide along the path 412, the rollers 418 pushing the cylinder 404, thereby causing the syringe 400 to slide through the syringe holders 408, and causing the injection needle 406 to emerge from the hole 414. The top 422 does not touch the plunger 402, and so the injection needle 406 is being inserted into the injection site, without injecting the substance. The movement of the top 422 relative to the base 410 is resisted by the springs 428, causing the springs to be further stretched, thereby accumulating potential energy that will restore the top 422 to its original position when the palm is removed.

With reference to FIG. 4B, the palm continues to press on the palm receiving surface 424 of the top 422, thereby moving the cylinder 404 until it reaches the end of its travel caused by the arms 420. The travel caused by the arms 420 ends when the rollers 418 spread wider than the width of the top end of the cylinder 404. The rollers 418 are led by the arms 420, the arms 420 being led by the path 412. Further, because the rollers 418 have lost contact with the cylinder 404, further pushing of the top 422 will result in an inner surface of the top 422 pushing the plunger 402 into the cylinder 404.

With reference to FIG. 4C, the end of each arm 420 is guided by the paths 412, causing the rollers 418 to no longer contact the top the cylinder 404, while the inner surface of the top 422 pushes the plunger 402 into the cylinder 404, thereby causing injection of the substance into the injection site. Because of the cutouts 432 in the top 422, the syringe retrievers 434 do not restrict relative movement between the top 422 and the cylinder 404.

With reference to FIG. 4D, the top 422 has reached the lowest point in its travel, and the plunger 402 has reached the end of its travel within the cylinder 404, and the springs 428 have reached their maximum extension. As a result of the plunger 402 reaching the end of it's travel within the cylinder 404, the injection of the substance is completed.

With reference to FIG. 4E, while top 422 returns to its initial position, the syringe retrievers 434 reach the end of the cutouts 432 in the top 422, the syringe retrievers 434 thereby beginning retrieving the cylinder 404. When the syringe retrievers 434 begin to move, they will pull the cylinder 404, thereby pulling the needle 406 into the base 410.

With reference to FIG. 4F, when the pressure of the palm upon the palm receiving surface 424 of the top 422 is removed, the springs 428 are allowed to return their initial pre-loaded state. The contraction of the springs 428 drives the top 422 to return to its initial position. In this embodiment, a standard syringe can be used. Once the top 422 has returned to its initial position, the syringe retrievers 434 have reached the end of the cutouts 432 in the top 422, and consequently the syringe retrievers 434 have retrieved the cylinder 404, thereby pulling the needle 406 completely into the base 410.

### Simultaneous Multi-Jector

Immunization schedules for infants are recommended by both the Centers for Disease Control and the American Academy of Pediatrics. These immunization schedules recommend administration of multiple vaccinations, which require a sequence of injections during each of three office visits, the injections occurring at two, four, and six months of age, and at one year, and at 18 months of age. During each vaccination visit, an infant may receive from two to six injections. This may result in anxiety for both the parents and the child, before, during, and after the visits, which may also interfere with the relationship between the parents and the healthcare provider. Furthermore, this is thought to contribute to excessive anxiety in children upon entering a medical office, and may also contribute to tendency towards life-long needle-phobia and/or doctor phobia (“White Coat Syndrome”).

Beyond immunization schedules, there are other medical conditions that require administration of a variety of injectable medications. As presently administered, a sequence of such injections can result in excessive anxiety, discomfort, fear, and pain.

The palm-controlled injector of the invention enables simultaneous multiple injections, thereby reducing for the patient the time, anxiety, and discomfort due to the injections, as compared with performing the injections sequentially. The proposed injector includes features that activate the “pain gate” effect, and is consequently likely to inflict less pain as compared with known injectors. Simultaneous administration of multiple injections is also likely to reduce for parents and caregivers the anxiety and frustrations associated with the injections, as compared with performing the injections sequentially. Furthermore, the simultaneous administration performed by the injector of the invention will result in time saved per patient, both from actual administration of the injections simultaneously, and from the reduced time spent to overcome patient resistance and struggle typically associated with multiple injections, leading to substantially improved efficiencies in the operation of medical facilities.

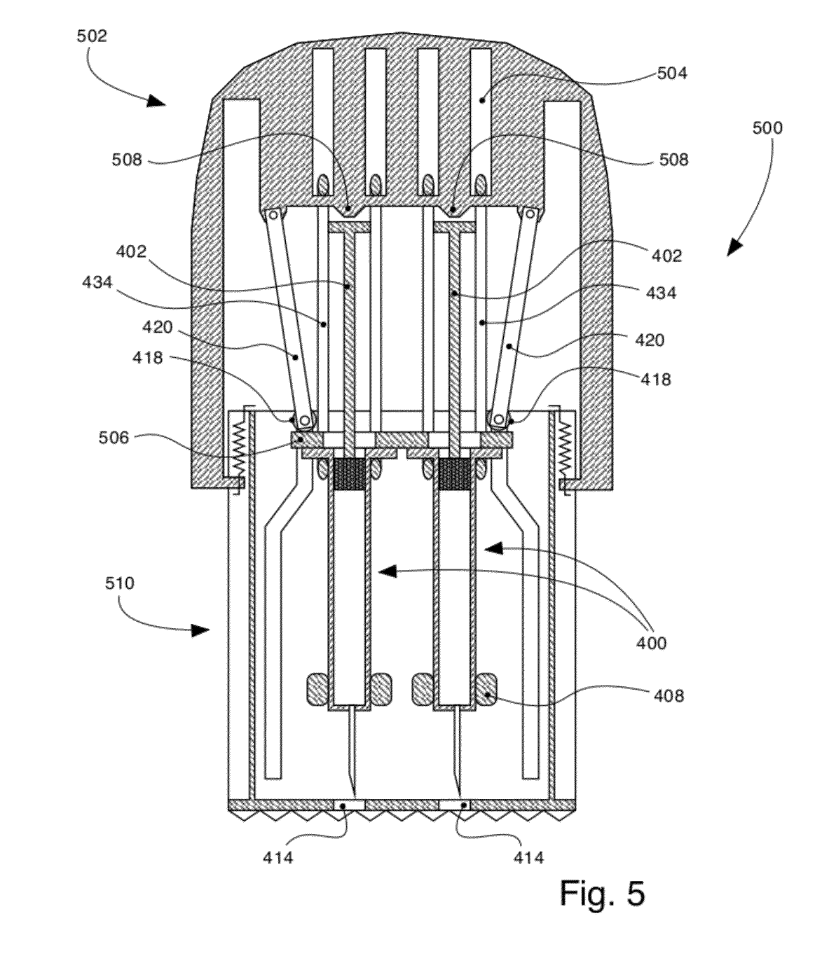

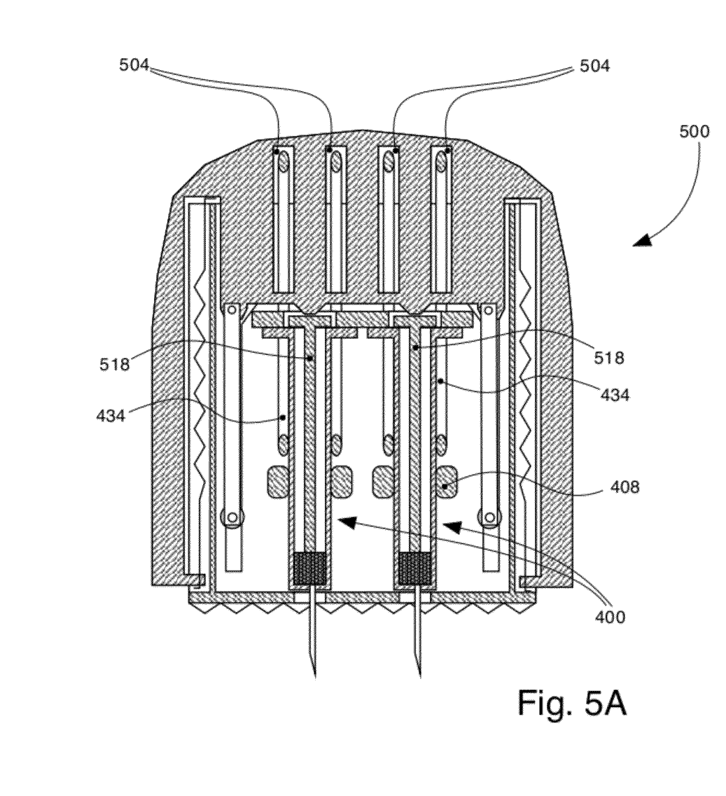

With reference to FIG. 5, the device 500 is similar to the device 430 shown in FIG. 4 in both structure and function, one difference being that device 500 can accommodate a plurality of syringes 400. Consequently, the top 502 has a plurality of pairs of cutouts 504 to accommodate a respective plurality of retrievers 434. Alternatively, the top 502 can have a plurality of single cutouts (not shown) to accommodate a respective plurality of retrievers (not shown), each retriever having two prongs to symmetrically pull each syringe 400, and a single prong to follow each single cutout (not shown). Another difference is that the arms 420, that hold the rollers 418, push upon a plate 506 that in turn pushes each of the syringes 400. The plate 506 includes a plurality of openings, each opening allowing a respective plunger to move unrestrictedly. The inner surface of the top 502 includes a plurality of bumps 508 capable of pushing respective plungers 402 unrestrictedly through the openings in the plate 506. Also, the base 510 includes a plurality of openings 414, to accommodate the respective plurality of syringes 400. Yet another difference, unrelated to the fact that the device 500 can accommodate a plurality of syringes 400, is that the cutouts 504 are shorter than the cutouts 432 shown in FIG. 4. Further, the retrievers 434 must travel along the length of the syringes 400 to accommodate for lesser travel range in each of the cutouts 504.

With reference to FIG. 5A, at the end of the full travel range of the device 500, each retriever 434 resides at the top end of the respective cutout 504, and each retriever 434 slides along the respective syringe 400 so as to accommodate for the lesser travel range in each of the cutouts 504. Further, the syringe holders 408 are located so as to not interfere with the travel of the retrievers 434.

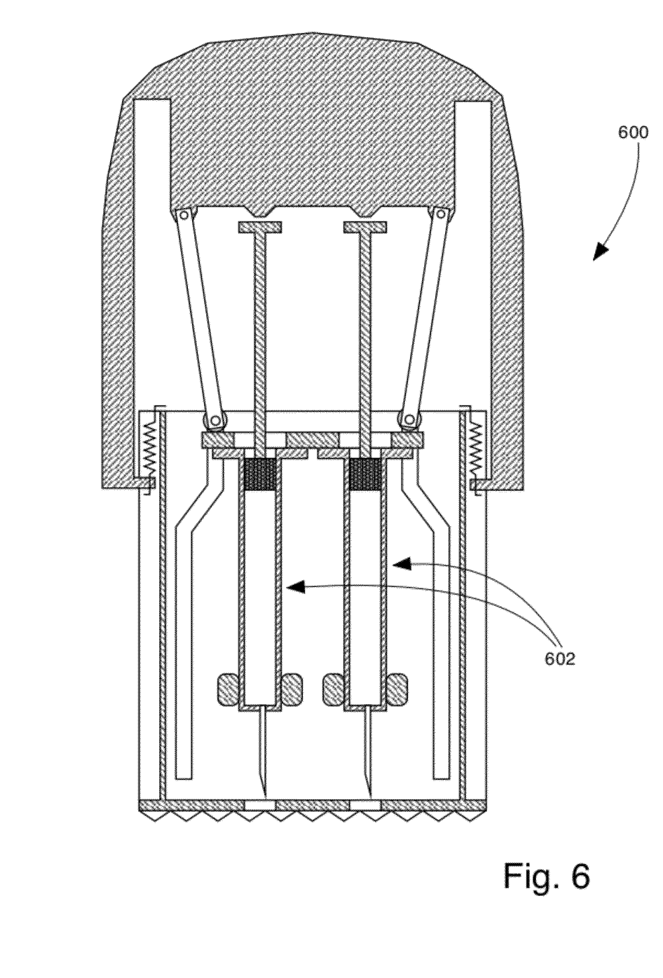

With reference to FIG. 6, the device 600 is similar to the device 500 shown in FIG. 5 in both structure and function, one difference being that device 600 does not include any retrievers 434, and does not include any cutouts 504. In this embodiment, we recommend using syringes that automatically withdraw the injection needle into each syringe after injection is completed.

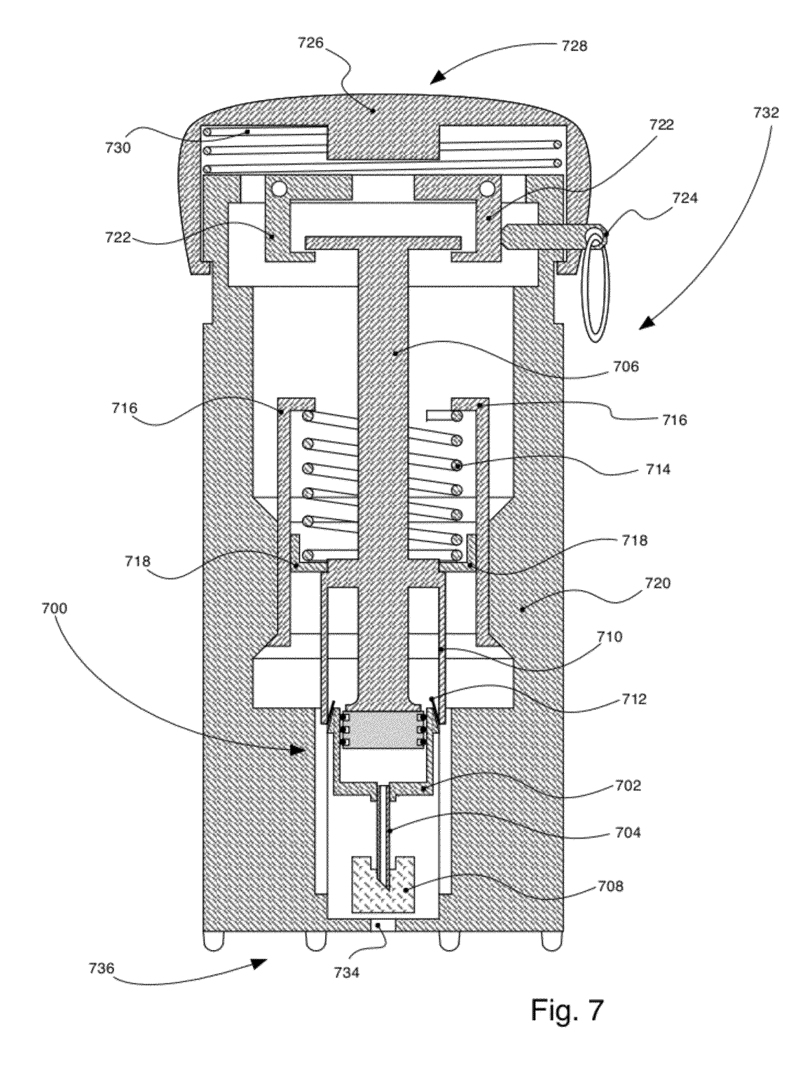

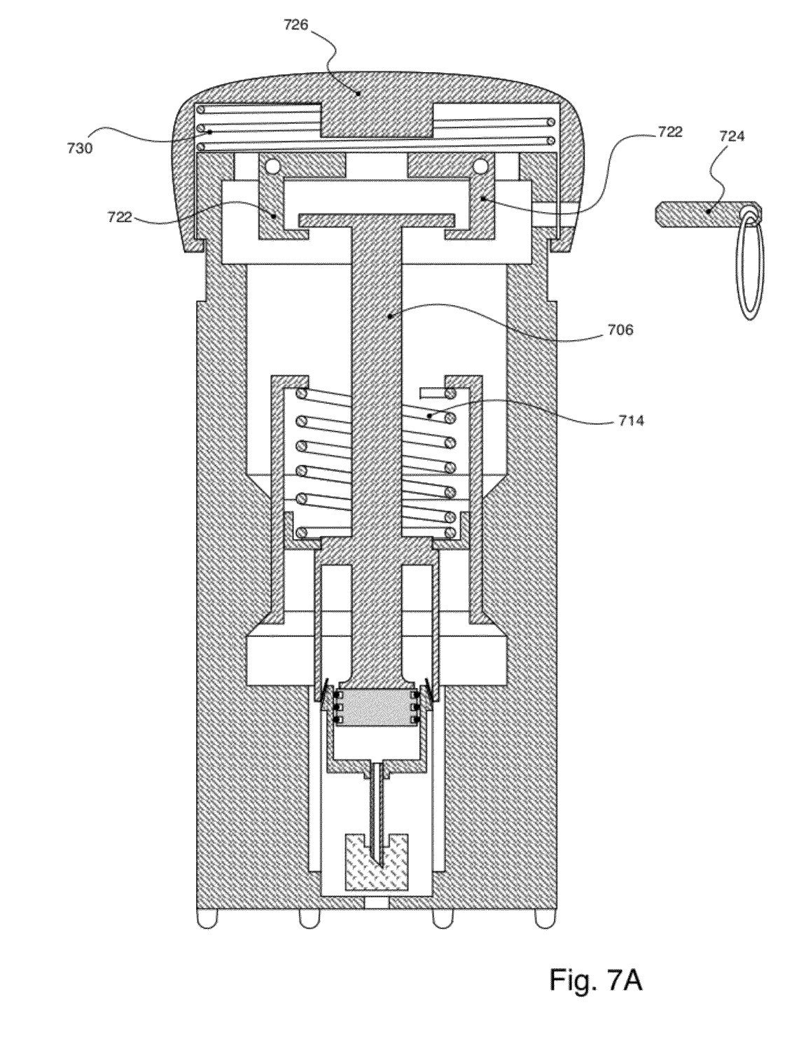

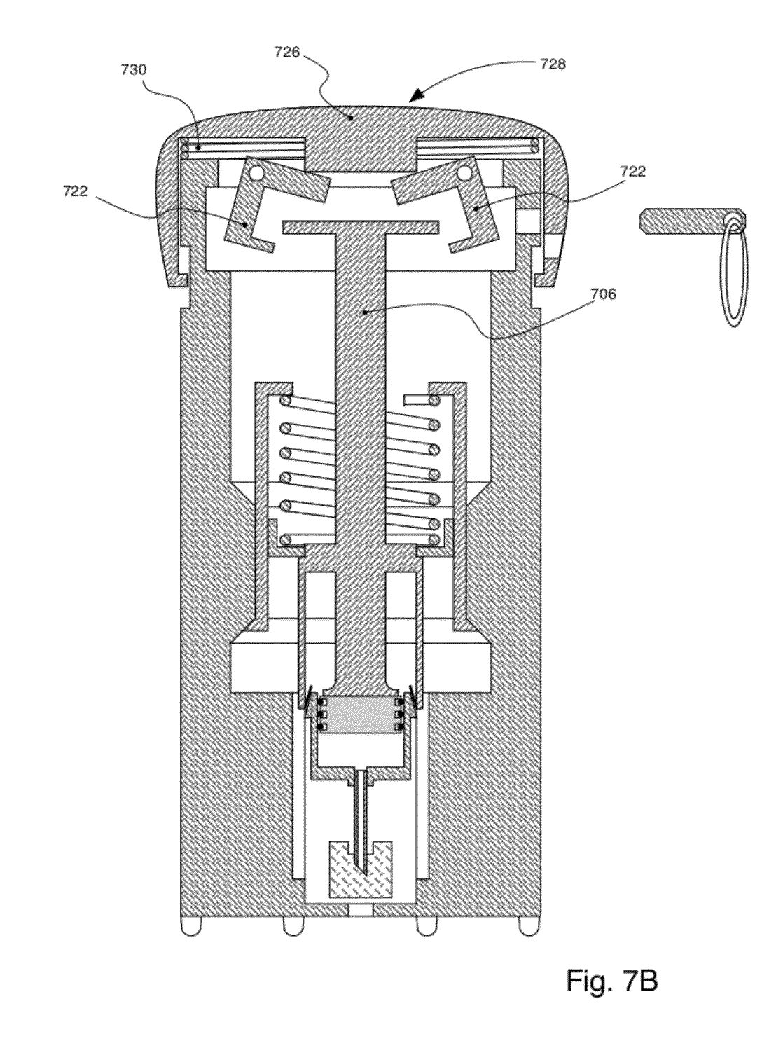

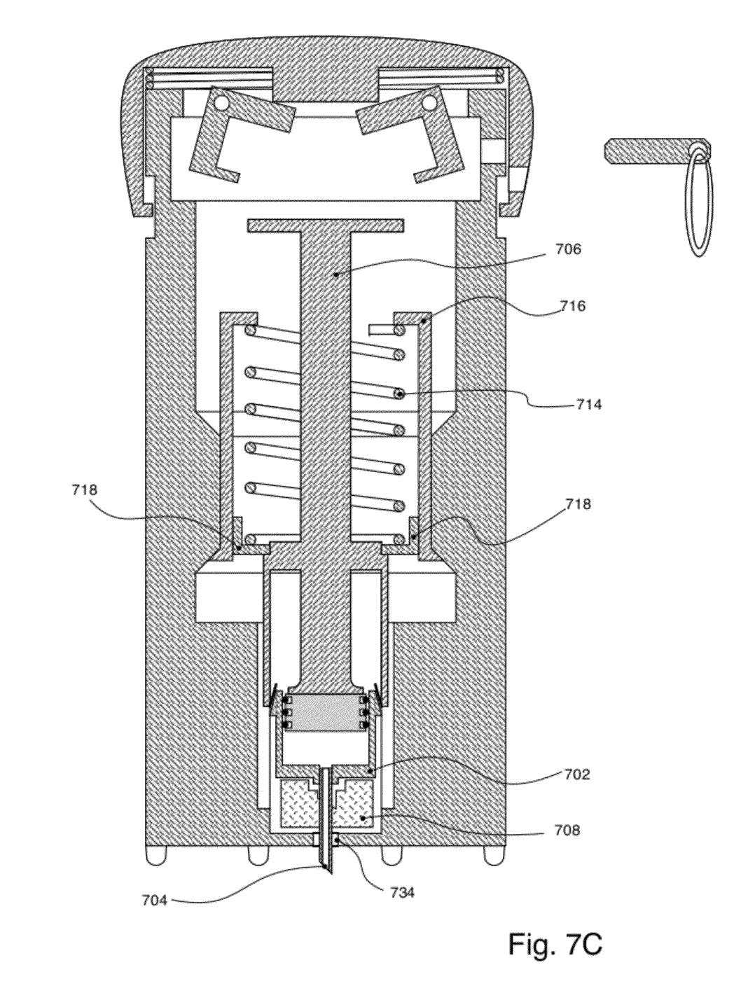

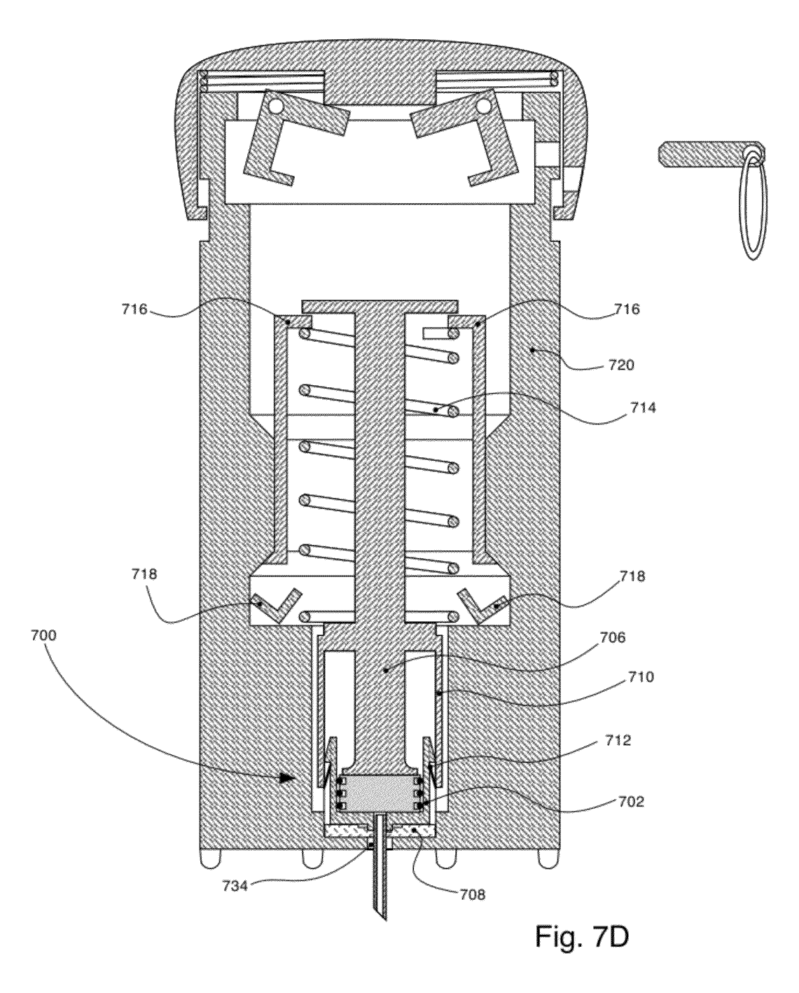

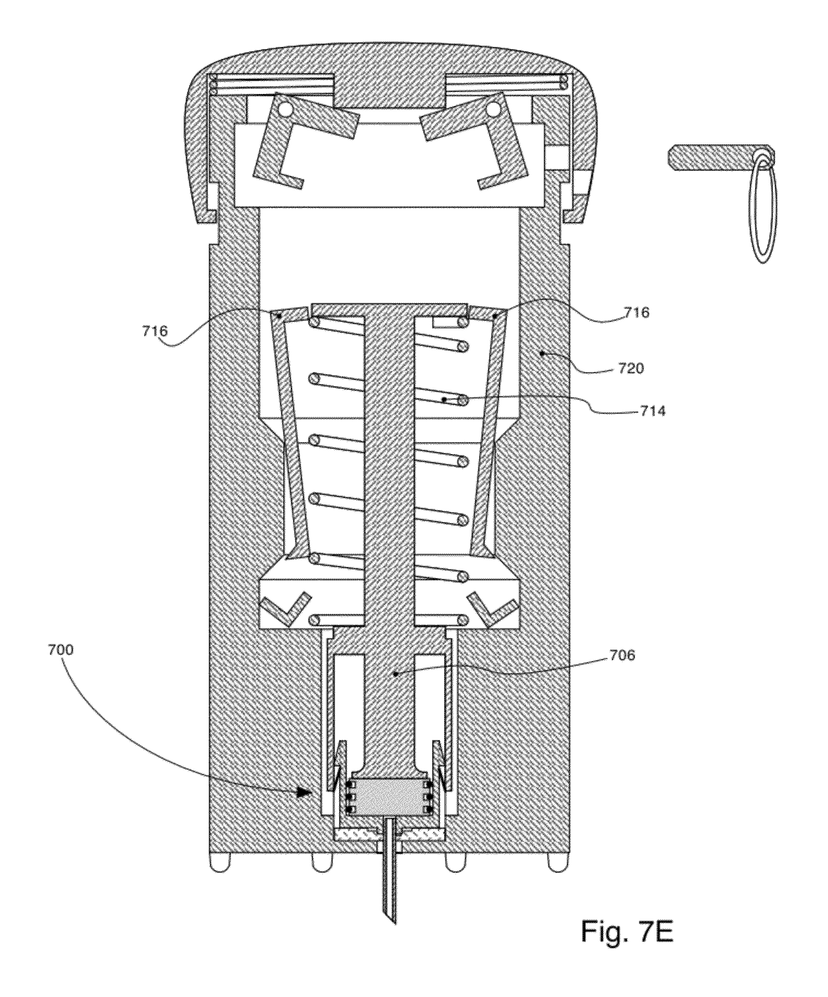

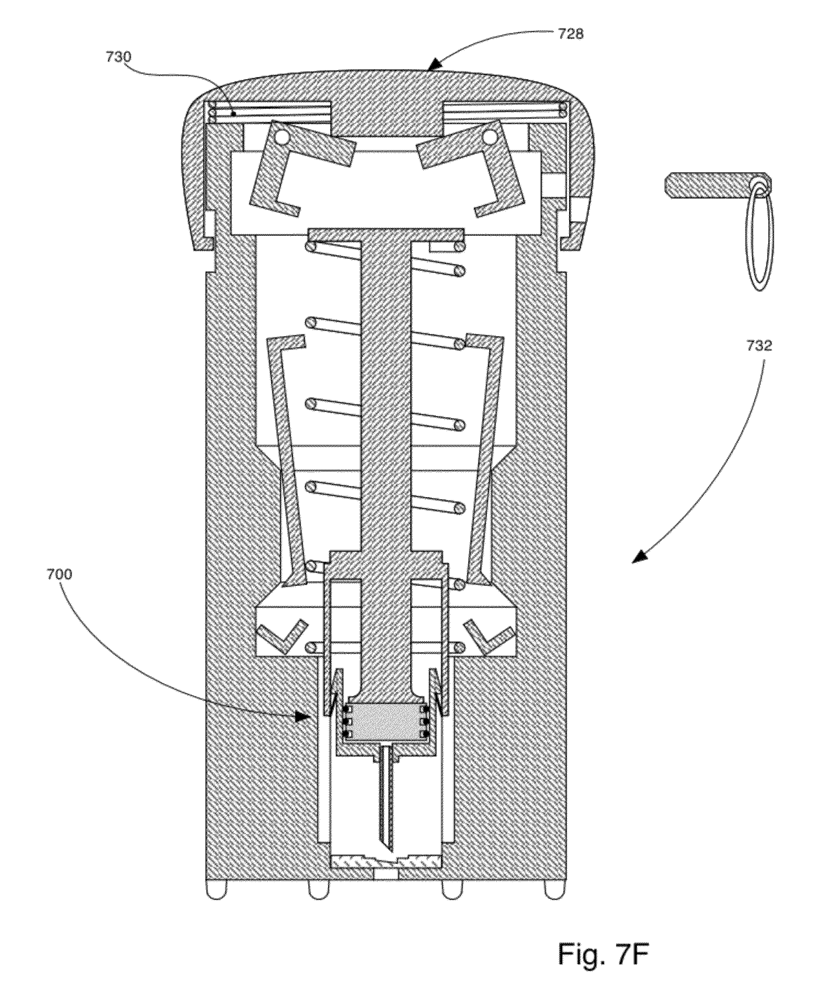

With reference to FIG. 7, a syringe 700 has a cylinder 702 containing a substance to be injected, and has an injection needle 704 and a plunger 706. The sharp end of the injection needle 704 is protected by a protective barrier 708 that prevents the substance from leaking out of the syringe 700. The protective barrier 708 also maintains the injection needle 704 in a clean condition. The protective barrier 708 also prevents the cylinder 702 and the injection needle 704 from accidentally separating from the plunger 706, thereby inadvertently exposing the injection needle 704.

The plunger 706 has arms 710 with latching springs 712. When the plunger 706 travels fully into the cylinder 702, the latching springs 712 latch onto the cylinder 702, so as to ensure that the plunger 706, the cylinder 702, and the injection needle 704 move together during retraction of the syringe 700.

The top of a spring 714 presses against the top portion of the spring retainers 716, while the bottom of the spring 714 presses against the plunger reversal brackets 718. Each plunger reversal bracket 718 leans against the plunger 706, and leans against a respective spring retainer 716, thereby preventing the spring retainers 716 from moving inwards. The inner surface of the body 720 is shaped so as to prevent the spring retainers 716 from moving upwards unless the spring retainers 716 can also move inwards. The spring retainers 716 cannot move inwards, and therefore cannot move upwards, because the plunger reversal brackets 718 block inwards movement of the spring retainers 716. The pressure exerted by the preloaded spring 714 against the plunger reversal brackets 718 resting on a ledge of the plunger 706 stabilizes the plunger reversal brackets 718 and the spring retainers 716, while allowing a mutually sliding relationship between the plunger reversal brackets 718 and the spring retainers 716.

The pre-loaded spring 714 would cause the plunger 706 and the plunger reversal brackets 718 to slide along the spring retainers 716, but for the swivel releases 722 that prevent the plunger 706 from moving.

A safety 724 prevents a top trigger 726 having a palm receiving surface 728 from compressing a safety spring 730, and then causing the swivel releases 722 to release the plunger 706 to move in response to the pressure exerted by the preloaded spring 714.

Pressure upon the palm receiving surface 728 thus causes the device 732 to initiate insertion of the injection needle 704 through the hole 734 and into an injection site, and then to further inject the substance into the injection site, followed by automatic retraction of the injection needle 704 back into the body 720. Additionally, pressure upon the palm receiving surface 728 causes the body 720 to press the pain gating elements 736 against the periphery of the injection site, thereby activating a pain gate effect that reduces discomfort associated with the injection.

With reference to FIG. 7A, removing the safety 724 allows the top trigger 726 to compress the safety spring 730, compression of the safety spring 730 allowing the top trigger 726 to cause the swivel releases 722 to release the plunger 706 so that the plunger 706 can move in response to the pressure exerted by the preloaded spring 714.

With reference to FIG. 7B, pressure of a palm upon the palm receiving surface 728 caused the top trigger 726 to compress the safety spring 730, and causes the top trigger 726 to press upon the swivel releases 722 so as to release the plunger 706.

With reference to FIG. 7C, the top of the spring 714 presses against the top portion of the spring retainers 716, while the bottom of the spring 714 presses against the plunger reversal brackets 718. The plunger reversal brackets 718 press against the ledge of the plunger 706, causing movement of the plunger 706. Movement of the plunger 706 causes cylinder 702 to move towards the hole 734, also causing the injection needle 704 to move through the hole 734, after penetrating through the protective barrier 708. Due to natural viscosity and lack of compressibility of the liquid substance in the cylinder 101, pressing on the plunger 706 causes the cylinder 702 to move towards the hole 734, along with the injection needle 704, thereby causing the injection needle 704 to move through the protection barrier 708 and then through the hole 734. Once the injection needle 704 emerges from the hole 734, it begins to enter the injection site of the organism.

With reference to 7D, the cylinder 702 is shown reaching the end of its travel within the body 720, thereby compressing the protective barrier 708, and the plunger 706 is shown reaching the end of its travel within the cylinder 702. While the plunger 706 moves inside the cylinder 702, the latching arms 710 move along the outside of the cylinder 702. Before the plunger 706 reaches the end of its travel with the cylinder 702, the latching spring 712 of each latching arm 710 latches onto the cylinder 702 so as to cause the cylinder to move away from the hole 734 when the plunger 706 moves away from the hole 734 during retraction of the syringe 700.

When the spring 714 pushes the plunger reversal brackets 718 past the edge of the spring retainers 716, the spring retainers 716 no longer hold the plunger reversal brackets 718 in place, thereby causing the plunger reversal brackets 718 to be pushed out of place by the spring 714. When the spring reversal brackets 718 fall out of place, the bottom of the spring 714 no longer pushes on the plunger, instead pushing upon a confronting inner surface of the body 720.

With reference to FIG. 7E, the top of the spring 714 pushes the spring retainers 716 up and away, thereby allowing the top of the spring 714 to push against the top of the plunger 706. Pressure exerted by the spring 714 upon the confronting inner surface of the body 720, and upon the top of the plunger 706 causes retraction of the syringe 700.

With reference to FIG. 7F, the device 732 is shown in a retracted state, after both injection of the substance by the syringe 700, and the subsequent retraction of the syringe 700. The safety spring 730 can remain compressed due to pressure upon the palm receiving surface 728 during both injection and retraction. Alternatively, momentary pressure upon the palm receiving surface 728 can serve to trigger the device 732, thereafter allowing the safety spring 730 to be in an expanded state during both injection and retraction.

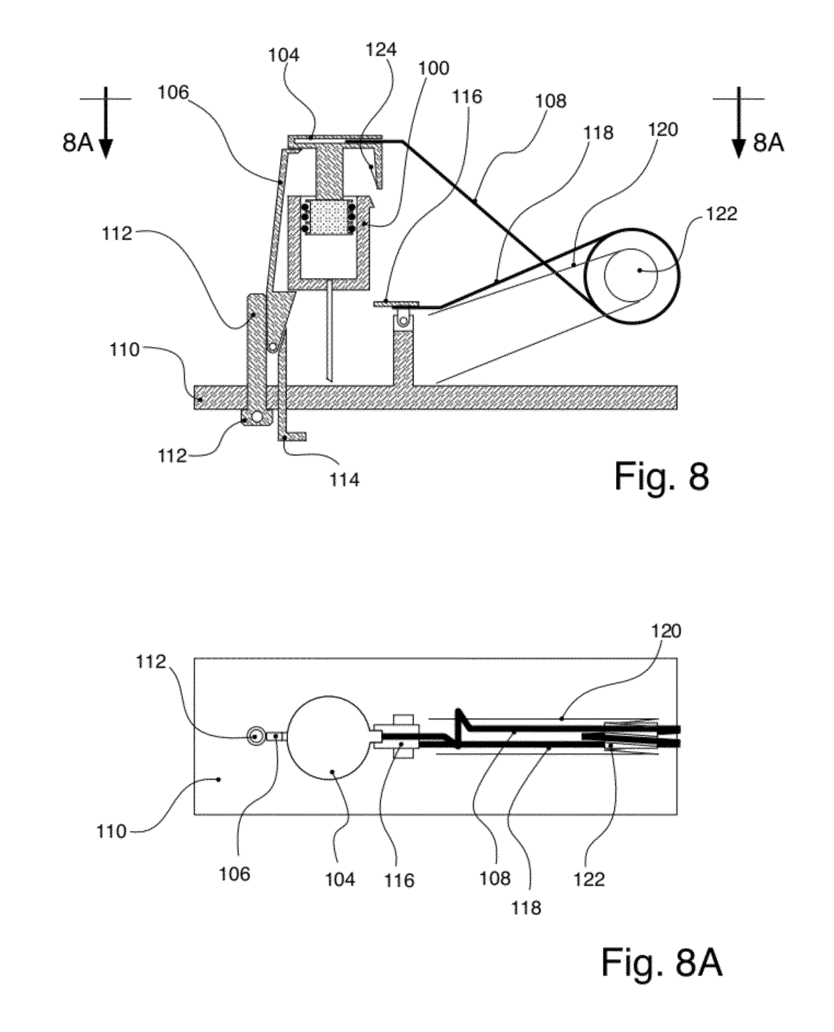

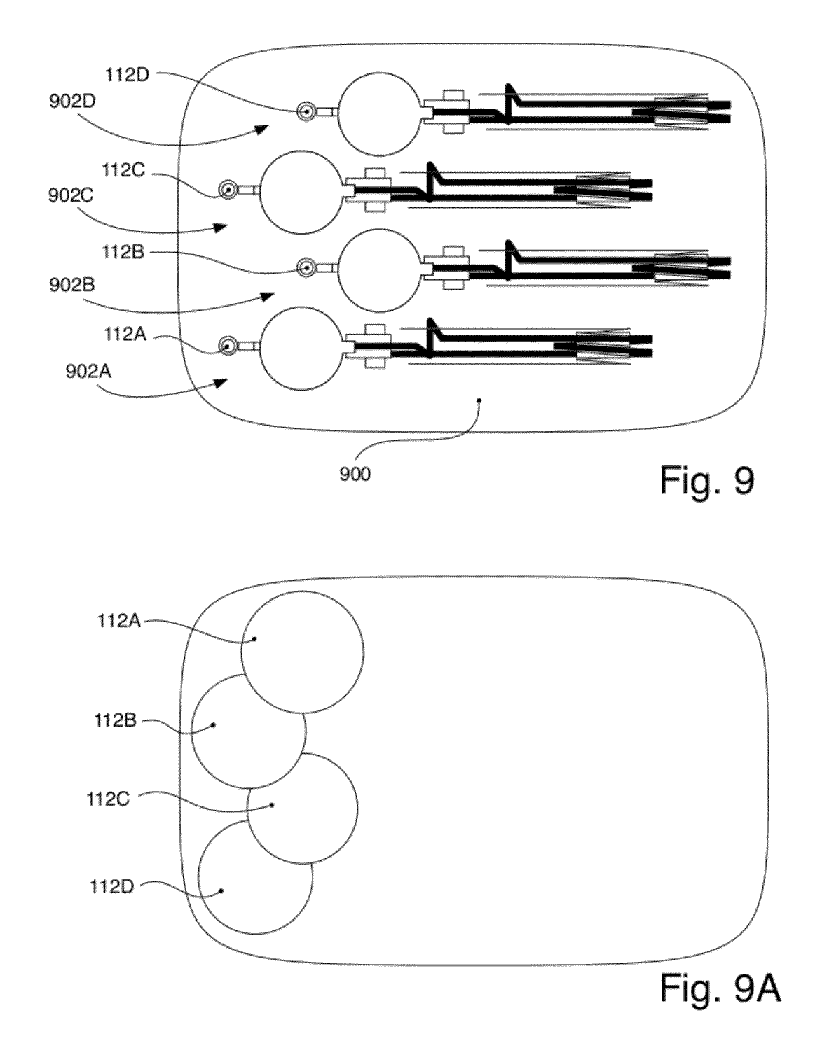

With reference to FIG. 8, the mechanism as described in FIG. 1 is shown as a single automatic injector for use in a multi-dose automatic injector, as shown in FIG. 9, and described herein below.

With reference to FIG. 8A, a top view of the single automatic injector of FIG. 8 is shown.

### Sequential Multi-Jector

Known emergency auto-injectors can include up to two doses of a single medication. However, at times, a need may arise for administration of more than two doses of the medication. For example, patients with food allergies may require more than two doses of epinephrine for multiple occurrences of an allergic reaction. Currently, patients are advised to carry two EpiPens® or one TwinJect® having two doses of epinephrine at all times. However, while a patient is on a flight, for example he/she may react to two different foods at two respective times during the flight, and so he/she may require more than two doses of epinephrine. Also, parents with multiple children, more than one having food allergies, can benefit from a single device with more than two doses of epinephrine.

The co-administration of a pair of medications is a common occurrence, such as the co-administration of antihistamine with epinephrine. The auto-injector of the invention can be used so as to administer multiple paired doses of different medications. Thus, if a patient with multiple food allergies experiences a sequence of allergic reactions during a flight, and consequently requires co-administered injections of both antihistamine and epinephrine, the emergency auto-injector of the invention can provide a plurality of co-administered doses.

With reference to FIG. 9, body 900 contains four separately operated automatic injectors 902A, 902B, 902C, and 902D, each as described in FIG. 8 and FIG. 8A, arranged so as to minimize required space within the body 900. Removal of a safety pin 112A of the injector 902A, enables removal of the safety pin 1128 of the second automatic injector 902B. Removal of a safety pin 1128 of the injector 902B, enables removal of the safety pin 112C of the third automatic injector 902C. Removal of a safety pin 112C of the injector 902C, enables removal of the safety pin 112D of the fourth automatic injector 902D.

With reference to FIG. 9A, the bottom of the body 900 is shown, so as to show the bottom of each of the four safety pins 112A, 1128, 112C, 112D. The bottoms of each of the safety pins 112A, 1128, 112C, 112D overlap, so as to enforce the sequential enablement of actuation of the plurality of automatic injectors 902A, 902B, 902C, and 902D.

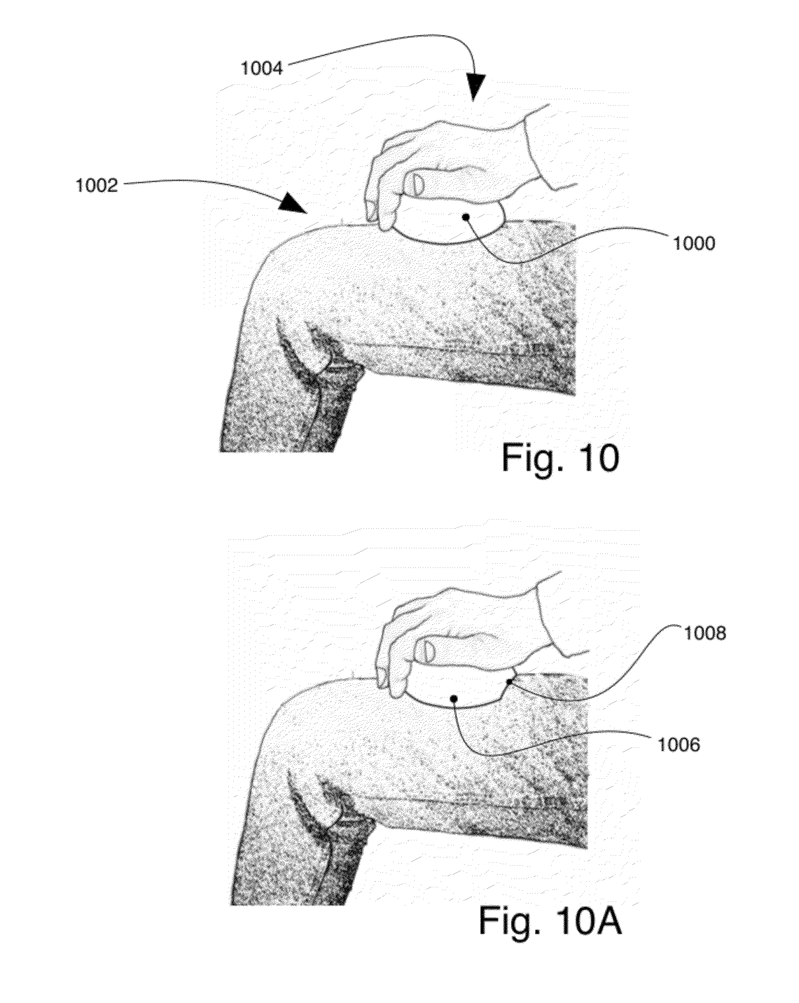

With reference to FIG. 10, an automatic palm activated injector 1000 is held in place on a thigh 1002 by a palm of a hand 1004.

With reference to FIG. 10A, an automatic palm activated injector 1006 has a concave injection surface 1008 that fits more closely to a convex injection site than a flat injection surface, as shown in FIG. 10.

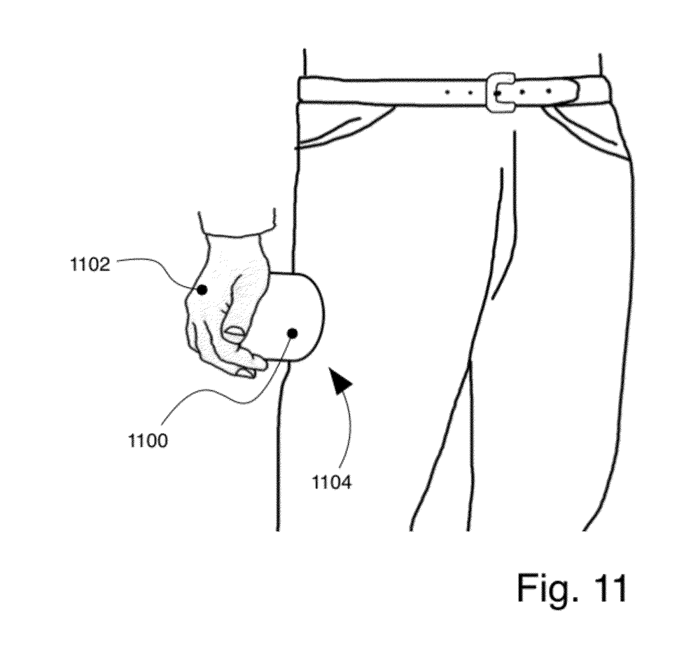

With reference to FIG. 11, a person is shown applying an automatic palm activated injector 1100 with a palm of a hand 1102 to a thigh 1104 by holding and slightly pressing upon the injector 1100 with a palm of the hand 1102 in substantially parallel relationship with respect to an injection site of an organism while operating the device.

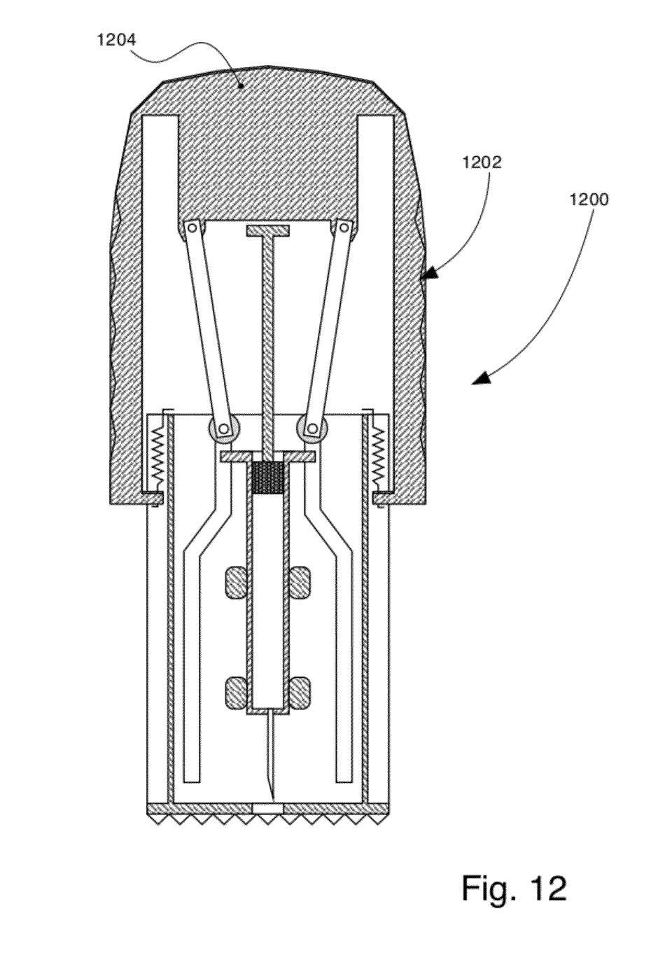

With reference to FIG. 12, a device 1200 is shown, similar to the device 330 shown in FIG. 3, except that the palm receiving surface 1202 of the top 1204 is shaped so as to receive a palm in substantially perpendicular relationship with respect to an injection site of an organism while operating the device.

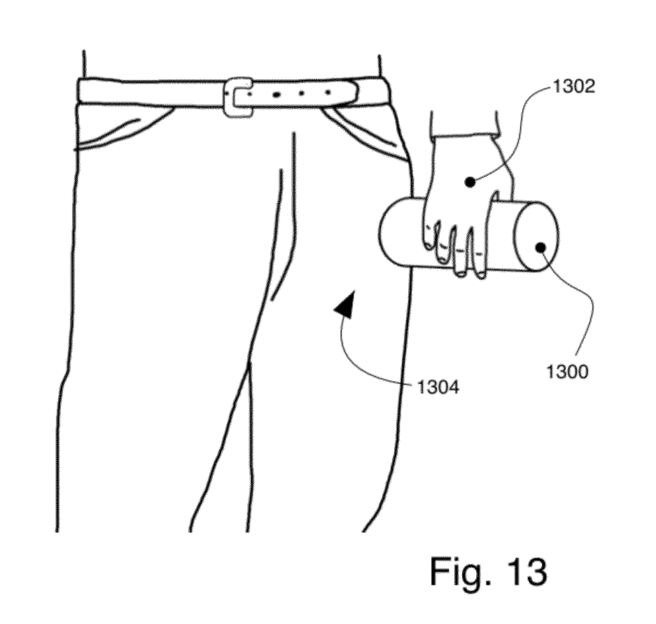

With reference to FIG. 13, a person is shown applying an automatic palm activated injector 1300 with a palm of a hand 1302 to a thigh 1304 by holding and slightly pressing upon the injector 1300 with a palm of the hand 1302 in substantially perpendicular relationship with respect to an injection site of an organism while operating the device.

Other modifications and implementations will occur to those skilled in the art without departing from the spirit and the scope of the invention as claimed. Accordingly, the above description is not intended to limit the invention, except as indicated in the following claims.

What is claimed is:

1. A palm-controlled device for injection of a substance into an organism, the device comprising:
a palm-receiving surface for receiving a palm of a hand, the palm receiving surface being shaped so that the palm is substantially parallel to a surface of an injection site of the organism while operating the device; and
an injector having at least one pre-filled syringe, the palm-receiving surface being cooperative with the injector such that when pressure is applied to the palm-receiving surface, the injector is actuated so as to inject contents of the at least one pre-filled syringe into the injection site of the organism.

2. The palm-controlled device of claim 1, wherein the palm-receiving surface is ergonomically shaped so as to receive at least a palm.

3. The palm-controlled device of claim 1, wherein the injector has an injector base through which the needle is moved when performing an injection.

4. The palm-controlled device of claim 3, wherein the injector base includes a concave surface which substantially conforms to a convex surface including the injection site.

5. The palm-controlled device of claim 3, wherein the injector base include features for activating a “pain gate” prior to penetration of the injection needle into the injection site.

6. The palm-controlled device of claim 5, wherein the features for activating a “pain gate” include at least one of:
a plurality of protrusions extending out from the surface of the injector base so as to provide a plurality of localized pressure areas to a region surrounding an injection site before and during penetration of the needle into the injection site;
at least one annular ridge of the surface of the injector base so as to provide at least one localized pressure area to a region surrounding at least one injection site before and during injection of at least one needle into at least one respective injection site; and
a substantially flat surface of the injector base so as to provide a continuous area for applying pressure to a region surrounding an injection site before and during injection into the injection site.

7. The palm-controlled device of claim 6, wherein the plurality of protrusions include at least one of:
balls, cubes, prisms, pyramids, bumps, cylinders, blunt probes.

8. The palm-controlled device of claim 6, wherein the plurality of protrusions are arranged in at least one of:
a regular array;
a random spatial distribution;
a concentric arrangement.

9. The palm-controlled device of claim 1, wherein the injector is capable of injecting at least one pre-loaded substance.

10. The palm-controlled device of claim 1, wherein the injector is capable of injecting a plurality of pre-loaded substances, the injecting occurring sequentially or simultaneously.

11. The palm-controlled device of claim 1, wherein the injector can automatically retrieve the needle after injection of the substance.

12. The palm-controlled device of claim 1, wherein the injector can include at least one safety mechanism for ensuring safe operation of the device.

13. The palm-controlled device of claim 1, wherein the injector includes a plurality of safety mechanisms, operation of each safety mechanism allowing injection of a single substance corresponding to a respective safety mechanism.

14. The palm-controlled device of claim 1, wherein the injector includes an element for transmitting manual pressure applied by at least the palm of the hand to a syringe including the injection needle so as to cause the injection needle to penetrate into the injection site.

15. The palm-controlled device of claim 1, wherein the injector includes an element for transmitting manual pressure to a trigger that allows automatic force to be applied to a syringe including the injection needle so as to cause the injection needle to penetrate into the injection site.

16. The palm-controlled device of claim 1, wherein the injector includes an element for transmitting manual pressure applied by at least the palm of the hand to a syringe including a plunger so as to cause the plunger to inject the substance into the injection site.

17. The palm-controlled device of claim 1, wherein the injector includes an element for transmitting manual pressure to a trigger that allows automatic force to be applied to a plunger so as to cause the plunger to inject the substance into the injection site.

18. The palm-controlled device of claim 1, wherein injection of a substance follows after penetration of the injection needle.

19. The palm-controlled device of claim 1, wherein injection of a substance proceeds while penetration of the injection needle proceeds.

20. The palm-controlled device of claim 1, wherein the injector includes:
a signaling element capable of indicating when a complete dose of a substance has been injected, by at least one of: visual signaling, audible signaling, tactile signaling.

21. The palm-controlled device of claim 1, wherein the injector includes:
apparatus capable of enabling both installing and removing a pre-filled syringe including the injection needle.

22. The palm-controlled device of claim 1, wherein the injector includes:
apparatus capable of automatically withdrawing the injection needle back into the injector after injection of the substance.

23. The palm-activating device of claim 22,
the injector including a syringe having the injection needle, a cylinder, and a plunger,
the apparatus including an element that interlocks the plunger with the cylinder so as to enable withdrawal of the injection needle by simultaneous withdrawal of the plunger and the cylinder.

24. The palm-controlled device of claim 1, the injector including a syringe having a retractable injection needle that is automatically retracted into the syringe after injection of the substance.

25. A palm-controlled device for injection of a substance into an organism, the device comprising:
a palm-receiving surface for receiving a palm of a hand, the palm receiving surface being shaped so that the palm is substantially parallel to a surface of an injection site of the organism while operating the device; and
an injector having apparatus capable of accepting and releasing at least one pre-filled syringe, the palm-receiving surface being cooperative with the injector such that when pressure is applied to the palm-receiving surface, the injector is actuated so as to inject contents of at least one accepted pre-filled syringe into the injection site of the organism.

26. The palm-controlled device of claim 25, wherein the palm-receiving surface is ergonomically shaped so as to receive at least a palm.

27. The palm-controlled device of claim 25, wherein the injector has an injector base through which the needle is moved when performing an injection.

28. The palm-controlled device of claim 27, wherein the injector base includes a concave surface which substantially conforms to a convex surface into which the injection needle is to be injected.

29. The palm-controlled device of claim 27, wherein the injector base include features for activating a “pain gate” prior to injection of the injection needle.

30. The palm-controlled device of claim 25, wherein the injector is capable of accepting at least one pre-loaded syringe.

31. The palm-controlled device of claim 25, wherein the injector can automatically withdraw the needle after injection of the substance.

32. The palm-controlled device of claim 25, wherein the injector includes an element for transmitting manual pressure applied by at least the palm of the hand to the pre-filed syringe including the injection needle so as to cause the injection needle to penetrate into the injection site.

33. The palm-controlled device of claim 25, wherein the injector includes an element for transmitting manual pressure applied to a trigger that allows automatic force to be applied to the pre-filled syringe including the injection needle so as to cause the injection needle to penetrate into the injection site.

34. The palm-controlled device of claim 25, wherein the injector includes an element for transmitting manual pressure applied by at least the palm of the hand to a syringe including a plunger so as to cause the plunger to inject the substance into the injection site.

35. The palm-controlled device of claim 25, wherein the injector includes an element for transmitting manual pressure applied to a trigger that allows automatic force to be applied to a plunger so as to cause the plunger to inject the substance into the injection site.

36. The palm-controlled device of claim 25, wherein injection of a substance follows penetration of the injection needle.

37. The palm-controlled device of claim 25, wherein injection of a substance proceeds while penetration of the injection needle proceeds.

38. The palm-controlled device of claim 25, wherein the injector includes:
a signaling element capable of indicating when a complete dose of a substance has been injected, by at least one of: visual signaling, audible signaling, tactile signaling.

39. A palm-controlled device for injection of a substance into an organism, the device comprising:
a palm-receiving surface for receiving pressure directly applied by at least the palm of a hand, the palm of the hand having a surface normal vector, the pressure being characterized by a pressure vector, the surface normal vector being substantially parallel to the pressure vector; and
an injector having an injection needle, the injector being characterized by an injection vector that is co-linear with the injection needle, the injection vector being substantially parallel to the surface normal vector.

40. A palm-controlled device for injection of a substance into an organism, the device comprising:
a palm-receiving surface for receiving pressure directly applied by at least the palm of a hand, the palm of the hand having a surface normal vector, the pressure being characterized by a pressure vector, the surface normal vector being substantially parallel to the pressure vector; and
an injector having apparatus capable of accepting and releasing at least one pre-filled syringe, the injector being characterized by an injection vector that is co-linear with an injection needle of the pre-filled syringe when accepted by the apparatus, the injection vector being substantially parallel to the surface normal vector.

41. A palm-controlled training device for teaching injection of a substance into an organism, the device comprising:
a palm-receiving surface for receiving a palm of a hand, the palm receiving surface being shaped so that the palm is substantially parallel to a surface of a potential injection site of the organism while operating the device; and
an injector having at least one dummy syringe, the palm-receiving surface being cooperative with the injector such that when pressure is applied to the palm-receiving surface, the injector is actuated so as to simulate injection of contents of the at least one dummy syringe into the potential injection site of the organism.

42. A method for injection of a substance into an organism, the method comprising:
using an open palm of a hand to activate an injector to inject the substance into an injection site of the organism.

43. The method of claim 42, wherein the palm is substantially parallel to a surface of an injection site of the organism.

44. The method of claim 42, wherein when the injector is activated, contents of at least one pre-filled syringe is injected into an injection site of the organism.

45. A palm-controlled device for injection of a substance into an organism, the device comprising:
a palm-receiving surface for receiving a palm of a hand, the palm receiving surface being shaped so that the palm is substantially perpendicular to a surface of an injection site of the organism while operating the device; and
an injector having apparatus capable of accepting and releasing at least one pre-filled syringe, the palm-receiving surface being cooperative with the injector such that when pressure is applied to the palm-receiving surface, the injector is actuated so as to inject contents of at least one accepted pre-filled syringe into the injection site of the organism.
